(19) United States
(12) Patent Application Publication
(10) Pub. No.: US 2006/0235280 A1
(43) Pub. Date: Oct. 19, 2006

(54) Health care management system and method

(76) Inventors: Glenn Vonk, Fuguay-Varina, NC (US); Richard Rumbaugh, Durham, NC (US); David Whellan, Bala Cynwyd, PA (US); Christopher O'Connor, Durham, NC (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 10/479,132

(22) PCT Filed: May 28, 2002
(86) PCT No.: PCT/US02/16629

§ 371 Date: Nov. 19, 2004

Related U.S. Application Data
(60) Provisional application No. 60/293,541, filed on May 29, 2001.

(51) Int. Cl. A61B 5/00; G06Q 10/00

(52) U.S. Cl. 600/300; 705/2

(57) ABSTRACT

An electronic health care management system is provided which collects both subjective and objective information regarding a patient into a clinical patient record, and uses the record to determine evidence-based recommendations. A healthcare provider may decide to implement certain recommendations, and/or provide additional interventions which are collectively implemented using automated support tools. Often, a plan can include follow-up activities which may be automatically scheduled by the electronic health care management system, and may include external scheduling programs and corresponding application-programming interfaces (APIs).

This application claims priority under 35 U.S.C. §119(e) from a U.S. provisional patent application of Glenn Vonk et. al., Ser. No. 60/293,541, filed May 29, 2001, entitled “E-Care Software for Disease Management”, the entire content of which is expressly incorporated herein by reference.

### FIELD OF THE INVENTION

The invention is related to healthcare management. More particularly, the invention is related to a system and method for integrating an Internet browser-based client and a database backend with patient monitoring devices to provide a complete feedback loop between the doctor, nurse, physician's assistant and patient.

### BACKGROUND OF THE INVENTION

Over 90 million Americans suffer from at least one chronic disease. The annual direct costs of diabetes, respiratory diseases, congestive heart failure, hypertension, and cancer come to over $148 billion. These conditions are responsible for significant morbidities including amputation, blindness, and lost productivity in addition to associated increases in mortality.

Healthcare providers have targeted diabetes and asthma, among others, as disease groups that would benefit from a new approach to patient care. The solution that most healthcare providers have identified is disease management. Already, 77 percent of Health Systems and Managed Care Organizations have programs in place, and 15 percent have programs in development. Most of the existing programs are for diabetes (74%), and the vast majority of disease management programs are less than two years old. While these institutions believe that improving the quality of care will drive down costs, they typically differ on how to measure the quality of their care. However, nearly all agree that they must be able to show short-term cost savings to justify the initial investment of money and hospital resources. The following are goals commonly cited by institutions when creating disease management programs:
Improve Outcomes—Improving the outcome of a disease means improving quality of life, reducing mortality, and improving clinical measures (for example, collecting information about disease progress and reaction to medication).
Improve Quality of Care—By improving physician adherence to best practices, and improving patient education and involvement in disease treatment, patients will receive better care.
Reduce Costs—Disease groups generate costs differently, but in all cases, hospital admissions and emergency room visits are significant and often preventable factors. By reducing unnecessary hospitalizations and emergency room visits, and by more effectively administering medications, organizations can immediately drive down costs associated with the disease.

Achieving these goals is in large part dependent on having a proven and repeatable set of protocols for diagnosing and treating the disease in question. These protocols must provide detailed, accurate guidance for treatment grounded in clinical evidence-based medicine. This allows physicians to stay up to date with the latest clinical research and more accurately prescribe and modify medication dosages. However, these protocols must also be flexible enough to allow for physicians or organizations to modify them based on their experience.

Another important success factor is patient monitoring and assessment, and education. Disease management relies on accurate patient information to alert doctors or medical staff to the beginning of a potentially serious condition (before that condition requires that the patient be hospitalized), to monitor drug effectiveness, and to produce clinical data Education allows patients to become part of the treatment process, and allows them to more properly channel their concerns about their health toward modifying their life-style and following their medication regimen.

The upper levels of hospital administration typically develop disease management programs. They usually face opposition both from finance departments that are concerned with up-front costs, and primary care physicians and specialists who are concerned about loss of control and “cookbook” medicine involving inflexible protocols that prevent them from using their own judgment when treating patients. Any successful disease management program must address these concerns or risk failure. In fact, in most organizations where disease management systems are in place, both physicians and administrators feel that their program could be vastly improved. However, because measures of success vary, there is often no consensus on how to improve them.

A significant part of the problem with many disease management programs lies in the basic way they are implemented. Approaches to disease management have typically centered on removing the patient monitoring and assessment workload from the congestive heart failure (CHF) treatment provider in one of the following ways:

1. Carve-out—Carve-out solutions usually involve farming out patient monitoring and assessment to a call center of trained nurses. These nurses then handle basic monitoring and assessment tasks, answer questions about medications and diet, and generally filter out calls that don't require the physician to treat the patient.
Benefits—This solution can reduce workload at the hospital, and doesn't require equipment or training investments by the provider.
Drawbacks—Obviously, this can be a source of frustration for the doctor, as the nurses at the call center are the ones that decide what information the doctor does, and does not, need to know. Physicians are reluctant to relinquish so much of their control.

2. Carve-In—Carve-in solutions are aimed at providing the same sort of information filtering as the carve-out solutions, but from within the hospital's existing support framework.
Benefits—This solution reduces the workload faced by nurses and doctors, and also provides more local control.
Drawbacks—Again, the intent of these programs is to put a layer of management between the doctor and patient. Because third party vendors staff these solutions, the doctors have once again put their patients in the hands of medical personnel with whom they are not used to working.

Both of these approaches create substantial problems. They interrupt and frequently supersede the traditional doctor-patient relationship, and are consequently resisted by the physician community. Furthermore, these solutions, involving third parties, often have financial incentive to keep patient calls and doctor referrals to a minimum, which patients find frustrating. The less likely patients are to use the system, the less good it does in terms of providing them a service, and in keeping unnecessary hospital visits down.

Just as importantly, neither of these solutions helps the physicians by providing evidence-based rules for diagnosis and treatment. These solutions are incomplete in that they focus primarily on guidelines, and fail to provide detailed tools to help the physicians improve patient care.

The central tenet of the system is that the physician must be in control. Physicians must not only buy into the idea of disease management for it to be effective; they must buy into the way it's being run. They are the ones ultimately responsible for their patient's care, so they must be given the tools to control that care.

Previous studies have identified benefits to management of chronic diseases such as CHF. For example, Rich in “Heart Failure Disease Management: A Critical Review” Journal of Cardiac Failure 1999, documents the value of a number of programs. Shulman and Bernard have obtained significant reductions in emergency room visits by asthma patients using disease management protocols at the University of Pennsylvania Medical Center. However, successes in disease management have been isolated and unconvincing. Few studies relate the high cost of delivering disease management solutions. Many disease management solutions are difficult to implement without significant disruption of normal clinic work practices. Finally, when a disease management program succeeds it is rarely exported to other healthcare systems. All these factors have limited the impact of disease management on healthcare practices.

Existing health-related software tools fall into several categories: (1) Electronic Medical Records (EMR), (2) Monitoring and surveillance software (3) electronic prescription software, (4) Routine “office” software, (5) Dedicated information systems (for example Laboratory Information Systems (LIS)). At best, each of these offers marginal benefits to healthcare providers. More commonly, these often create fragmentation and introduce additional complexities in clinical practice.

Known systems and methods thus have significant shortcomings in providing an integrated healthcare management system that addresses the needs of all the participants. These shortcomings can be summarized as follows:

(1) Managing Clinic Workflow. Presently, the majority of healthcare is provided through paper records. Complex care pathways are difficult to implement using paper records.

(2) Ineffective Electronic Data Gathering. Even when prior art healthcare management systems utilize electronic record keeping, their efforts are disjointed and ineffective. A typical clinic or home visit involves subjective and objective evaluations, assessment and plan of treatment. The subjective evaluation may be entered through automated web tools or voice annotation. Healthcare providers may obtain objective evaluations through automated devices at remote sites, or by manual entry of information, or through communication with an existing information system.

(3) Failure of Electronic Medical Records. Conceptually, an electronic mechanical archival (EMA) system archives every clinical observation or fact about a patient in an electronic format. Consequently, EMRs should improve access to medical information and enhance the quality of care. However, utilization of EMRs by healthcare providers has been very limited since (1) the large amount of data entry required created more work than the benefits warranted, and (2) data entry interferes with nominal clinic workflow.

(4) Liability of Unprocessed Information. Healthcare providers have legal liability for review of any patient information received, and for the actions they take or overlook. The potential data-flow may be large if a patient population is large, or engages in a large amount of monitoring. At some point, the data-flow may exceed the providers' capacity to review it. Providers will not accept data under these circumstances.

(5) Limited Utility of Monitoring Services. Monitoring services attempt to improve access to subjective and objective information though automation and transmission of information from remote sites to healthcare providers. Subjective information includes but is not limited to qualitative patient information about symptoms medications, diet, exercise, and quality of life. Objective information might include, but is not limited to, quantitative information about weight, blood pressure, pulse rate, blood glucose, and medications. These services offer value in the timeliness and quality of information critical to patient management. Typically, a surveillance function is offered to alert providers to patients that need attention. Monitoring services are valuable when used by trained healthcare providers that can evaluate patient data and determine appropriate interventions that both improve the patients' status and reduce costs. Many healthcare enterprises lack this expertise.

(6) Managing Complexity. Medicine is a complex undertaking, and medical research continues to add to this complexity at an accelerating pace. The number of pharmaceutical therapies is expected to rapidly increase due to the large number of therapeutic targets identified by genomics. In addition, genomics research will allow medications to be prescribed based on individual pharmacogenetic profiles. New tests, procedures and devices (including real time MRI Brain Natriuretic Peptide monitoring, continuous glucose monitoring/insulin delivery) will constitute important new diagnostic and therapy options. Layered on top of these issues is the complexity of managing comorbidities, all with similar increased complexities as described above. Maintaining awareness of new medical practice and customizing this knowledge to individual patients is difficult for healthcare providers. Optimal outcomes for patients and healthcare systems will be significantly improved by utilizing software to assist providers making complex clinical decisions.

(7) Customization to Local Practice Standards. Present software solutions do not attempt to incorporate new information. They utilize accepted guidelines at the time of their release. In addition, they do not attempt to individualize treatment plans or identify appropriate therapies based on clinical information. Patients and providers have unique and valuable perspectives on what constitutes good care within their communities. Although medical leaders and/or clinical trials may show a therapy to be effective, its adoption within a provider community may be slow. Potential reasons for slow adoption include difficulties disseminating trial results and/or the dissimilarity between trial patients and the provider's patients.

(8) Medical Errors. Present disease management solutions use simple guidelines to modify physician behavior without taking into consideration local practice standards or local patient characteristics. Patients become confused by the conflicting messages they are receiving from disease management companies and their physicians. Physicians become frustrated with inappropriate recommendations from the disease management companies and with angry patients who inappropriately blame their local providers for mismanagement.

Over 44 thousand Americans die each year as a result of medical errors during hospitalization (To Err is Human, Institute of Medicine, National Academy Press, Washington, D.C. 1999). Certainly, the issues of sub-optimal medical care become even more significant as healthcare moves away from traditional diagnosis and treatment of acute conditions. Many of these errors could be prevented using relatively simple interventions. In reality, few mechanisms exist for ongoing support of the chronically ill between acute episodes, or at remote sites.

(9) Economic Costs. U.S. healthcare costs totaled $1.2 trillion in 1999 (see, generally, data presented at “www.hcfa.gov”). Often, health systems provide care at low or negative margin. Preventive monitoring, patient support, and optimization of care can reduce total costs and provide significant economic benefits to payers and health delivery systems.

(10) Need for Quality Improvement. Healthcare systems are under constant pressure to improve clinical and economic outcomes. Although they are accountable by accreditation organizations to achieve process outcomes (an example of a process outcome would be the percentage of patients for which a glycated hemoglobin measurement was recorded in the previous year), these outcomes are limited in their impact.

Known methods have failed to resolve, either separately or wholly, the aforementioned problems in “integrated” healthcare management systems. Significant literature exits which describes there shortcomings, including two reports released by the Institute of Medicine titled “To Err is Human” (1999) and “Crossing the Quality Chasm” (2001) both published by National Academy Press (Washington D.C.). Benefits of intensive management of congestive heart failure have been summarized by Whellan et al. In “Disease Management of Congestive Heart Failure” American Journal of Managed Care 1999, 5(4), 499 and Rich in “Heart Failure Disease Management: A Critical Review” Journal of Cardiac Failure 1999, (5)1 64.

### SUMMARY OF THE INVENTION

The above described disadvantages are overcome and a number of advantages are realized by the present invention which relates to an enhanced fully integrated electronic healthcare management system.

It is therefore an object of the invention to collect both subjective and objective information regarding a patient into a clinical patient record (CPR) and use it to determine evidence-based recommendations. A healthcare provider may decide to implement certain recommendations, and/or provide additional interventions deemed necessary for the patient. These actions are collectively implemented using the automated support tools. Often, a plan will include future events such as a laboratory and clinic follow-up. An embodiment of the present invention is capable of automatically scheduling follow-up events when the healthcare provider decides to implement a particular plan. Where external scheduling programs and corresponding application-programming interfaces (APIs) are available, an embodiment of the present invention can schedule follow-up activities in the external systems. Examples of external scheduling systems include, but are not limited to laboratory services, consulting personnel, procedure teams and resources, referrals, billing and educational services. Providers may also experience enhanced personal satisfaction if the frequency of tedious and routine tasks is diminished. Thus, the present invention provides both clinical and economic efficiencies difficult to duplicate with manual systems.

It is an additional object of the invention to include only the minimum data required to address a majority of the conditions encountered for a particular patient. For example, a healthcare provider managing a patient with congestive heart failure (CHF) does not need tools to manage diabetes unless the patient has diabetes. If a patient has diabetes or is diagnosed with diabetes, the CPR of the present invention may optionally expand to address diabetes in addition to congestive heart failure. The CPR concept therefore scales with the complexity of the patient and imposes minimal complexity upon healthcare providers. Unlike EMRs, the CPR returns significant value for the time spent entering patient information.

It is a further object of the invention to address the issue of liability. First, the present invention provides a suite of productivity tools needed to manage this information using evidence-based practices. This includes identification of abnormal results. Second, the present invention supplies audit trails for all clinical decisions. These features address physician, healthcare system, and payer concerns regarding liability.

It is still further an object of the invention to incorporate many of the alerting and surveillance functions available in existing monitoring services. It allows healthcare providers to set alerts at levels appropriate to individual patients. It also provides modifiable protocols that produce a set of recommendations based on data from the monitors and the CPR. This assists providers with identification of well-defined interventions and reduces dependence on experts for relatively routine care scenarios. In particular, these features maximize the abilities of physician extenders to use monitoring information to advantage while working in cooperation with medical doctors.

It is yet an additional object of the invention to incorporate therapeutic protocols based on the most up-to-date clinical information. A patient's clinical data will be applied to these protocols and recommendations for that specific patient will be provided. An embodiment of the present invention will allow new protocols to be implemented. The present invention will test any new protocol for appropriate integration with pre-existing protocols prior to implementation. Because the system is provided in an application service provider model, updates will be offered to providers as information becomes available.

It is another object of the invention to enhance the doctor-patient relationship. In every case, medical doctors will determine the best treatment for their patients. An embodiment of the present invention enhances doctor-patient relationships by enabling physicians to bring best practices to their patients in a timely and cost effective venue.

It is yet a further object of the invention to allow the local providers to modify protocols to reflect their local practice standards. Once protocols are modified, the title invention will test all protocols within the system for appropriate integration prior to release. They will be allowed to turn off protocols that are not accepted within their community.

It is yet another object of the invention to address the problem of medical errors during hospitalization by offering decision support within the normal providers' workflow. The support is streamlined and offers considerable value for both preventing errors and optimizing care at remote sites. In addition, an embodiment of the present invention provides mechanisms for patient education and self-discovery.

It is a still further object of the invention to provide for incorporation of productivity tools to improve workflow that will shift resources away from low value interactions (which may not be reimbursed) and towards higher value interventions. Providers will be able to care for more patients and optimize clinic visits to achieve superior results.

It is yet another object of the present invention to include tools for monitoring clinical and economic outcomes. The evaluation tools address not only process outcomes, but also clinical outcomes (for example, % glycated hemoglobin at or below 7.0). This feedback enables health systems to make adjustments necessary to maintain superior clinical and economic performance in addition to accreditation requirements.

It is still another object of the invention to implement an integrated enhanced electronic healthcare management system which is comprised of a TCP/IP (Internet) telecommunications using an application service provider (ASP) model. The ASP model has several advantages over other models. First, the software is hosted and maintained at one or a few centralized locations. This reduces the complexity of maintenance. Also, centralization improves security, monitoring, and auditing of the software product's performance. Certain features available at the enterprise level (centralized) require technical administration or may not be possible at remote sites. At scale, hardware costs are also reduced.

It is therefore an object of the invent to integrate various features present in separate healthcare management systems into an integrated and enhanced environment which is optimized by a new feature, the care recommendation rules engine, which assists healthcare providers with multiple and complex functions. A system and method according to an embodiment of the present invention automates clinical practice and brings significant new efficiencies to healthcare enterprises.

The aforementioned objects of the invention, and others, are met by an embodiment of the present invention, which describes an automated disease management system for chronic diseases. The system builds on the success of existing programs by adding both a computer based system for providers to aid with patient monitoring and assessment, treatment implementation and scheduling, as well as electronic monitoring devices that can be used by patients in their homes.

### BRIEF DESCRIPTION OF THE DRAWINGS

The novel features and advantages of the present invention will best be understood by reference to the detailed description of the specific embodiments which follows, when read in conjunction with the accompanying drawings, in which:

FIG. 1 illustrates an electronic healthcare management system according to an embodiment of the present invention;

FIG. 2 illustrates a flow diagram of a method for enrolling a new patient and for entering new patient information into the electric care health management system in accordance with an embodiment of the present invention;

FIG. 3 illustrates a patient demographics used in an electronic care health management system in accordance with an embodiment of the present invention;

FIG. 4 illustrates a flow diagram of a method for creating and updating a patient record in an electronic care health management system in accordance with an embodiment of the present invention;

FIGS. 5A and B illustrate a flow diagram of a method for running a treatment plan in an electronic care health management system in accordance with an embodiment of the present invention;

FIG. 6 which illustrates a heart failure program test results data form used in an electronic care health management system in accordance with an embodiment of the present invention;

FIG. 7 illustrates a flow diagram of a method for calendar and event scheduling used in an electronic care health management system in accordance with an embodiment of the present invention;

FIG. 8 illustrates a telephone encounter form used in an electronic care health management system in accordance with an embodiment of the present invention;

FIG. 9 illustrates a flow diagram of a method for entering patient encounter data into a clinic patient record used by an electronic care health management system in accordance with an embodiment of the present invention;

FIG. 10 illustrates a doctor/physician extender bulletin board display interface for use in an electronic care health management system in accordance with an embodiment of the present invention;

FIG. 11 illustrates a flow diagram of a method for using the doctor/physician extender bulletin board in an electronic care health management system in accordance with an embodiment of the present invention;

FIG. 12 illustrates a physician extender's home page used in an electronic care health management system in accordance with an embodiment of the present invention;

FIG. 13 illustrates a patient record page used by an electronic care health management system in accordance with an embodiment of the present invention; and

FIG. 14 illustrates a patient interface used in an electronic care health management system in accordance with an embodiment of the present invention.

### DETAILED DESCRIPTION OF THE PREFERRED EMBODIMENT

The various features of the preferred embodiment will now be described with reference to the figures, in which like parts are identified with the same reference characters.

The components of an embodiment of the present invention will extend and improve the system's patient monitoring and assessment abilities by collecting electronically (and efficiently) the relevant data (blood pressure, weight, blood glucose, peak flow, among other data types), and the following chief benefits will be realized:

(1) Shift healthcare delivery away from the hospital, by improving patient education, and by offering electronic access to address basic patient needs, thereby further reducing costs while improving patient care;

(2) Reduce workload for nurses, physicians' assistants and doctors, by automating clinic scheduling, and placing part of the patient records on line, allowing organizations to more effectively manage resources and devote less time to paperwork; and

(3) Automate the accumulation of patient data, through the use of carefully conducted patient monitoring and assessment, which can be used to further improve protocols.

The core features of the program are:

(1) Evidence-based Medicine—All protocols are supported by strong clinical evidence. The protocols provide complete guidance for all symptoms, providing current medication and treatment guidance. The protocols can be modified to reflect the best practices of the Healthcare system, and their implementation is supervised by physicians, and coordinated by nurse practitioners (NP) or other physician extenders, such as specially trained nurses, clinical specialists, physicians' assistants (PAs) and so on.

(2) Increased Access to Healthcare—Patients should not have to fight through layers of bureaucracy to obtain information. This is typically part of the MCO strategy to reduce costs, but typically causes more hospitalizations, as patients don't get proper feedback or supervision. Access means that patients feel more comfortable about their care, and have better access to answers about their symptoms. Lack of access means that patients often feel that they have no choice but to visit the emergency room during any onset of symptoms.

(3) Patient Education—Patient education should not simply include instructions for medication, but should include lifestyle changes going forward throughout their treatment. Education should also continue beyond the doctor's office, and be offered as part of an ongoing program of disease management.

(4) Healthcare System Based—The entire program should be based within the healthcare system so that it is implemented by specially trained nurses, physicians' assistants and so on that the physicians know and trust.

An exemplary embodiment of the present invention is comprised of a secured TCP/IP (Internet) telecommunication interface using an application service provider model (ASP). This exemplary embodiment of one present invention is further characterized in that it allows operative variations and alternatives. This may include individual software servers installed at each user's site. This gives the individual site control over their physical system, but introduces significant complexity.

The underlying technical principles of the exemplary embodiment of the present invention allow for individual components to be built on well-established technical principles for scheduling and other routine office tasks. However, the software will exhibit significant flexibility to allow for automated entry of important events into scheduling tools (such as calendars/“to do” lists). Further improvements and recommendations will be based on evidence based practices. Much of medical practice relies on subjective interpretation of many clinical observations. The software tool of the present invention will only make recommendations based on well-studied and established clinical outcomes.

The exemplary embodiment of the present invention will also provide for the retrospective analysis of data from patient populations using the title invention. Analysis of management trends in these populations and associated outcomes could reveal completely unexpected factors affecting patient status. These discoveries could be analyzed, optimized, and transmitted, yielding further enhancements of clinical and economic outcomes, at an accelerated pace.

Additionally, the exemplary embodiment of the present invention will provide for the integration of cost data from healthcare systems with resource utilization data. Since healthcare systems consider cost information proprietary and are reluctant to release it, the present invention may provide economic outcomes based on resource utilization using estimated costs (as well as actual costs, if available) for those resources.

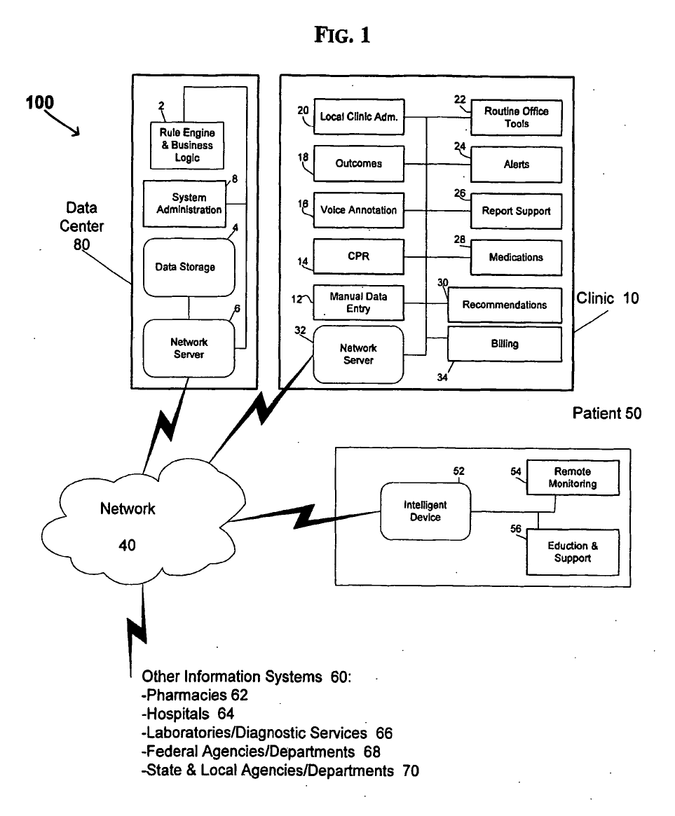

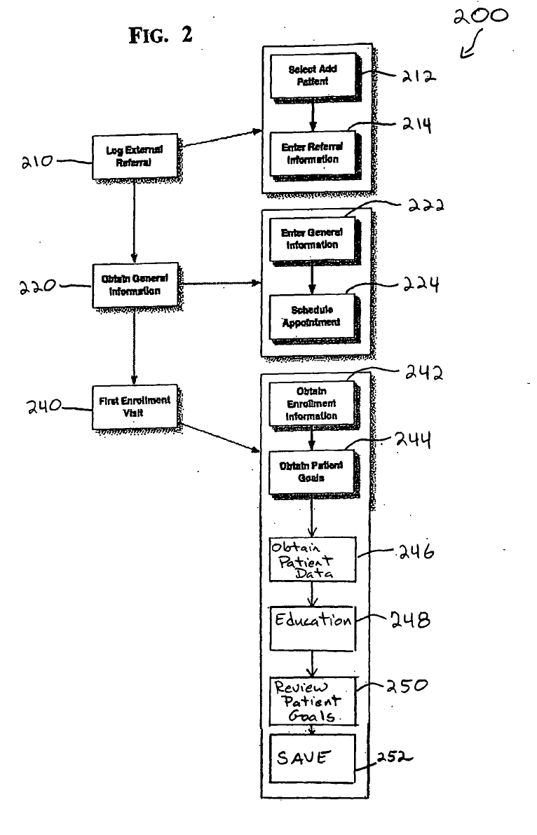

FIG. 1 illustrates an electronic healthcare management system according to an embodiment of the invention. The electronic healthcare management system (ECHMS) 100, of the invention, illustrated in FIG. 1, is comprised of a data center 80, clinic 10, patient 50, and other information systems 60 interconnected by network 40. Network 40 may be the Internet. Clinic 10 represents a facility in which patients will visit to see physicians extenders and/or doctors and/or nurses, and to receive treatment in the form of medication, evaluation, and/or therapy. Data center 80 is an administrative center that, preferably, stores or archives patient data (in the form of a clinic patient record, discussed in detail below), and essentially controls operation of electronic care health management system 100.

Data center 80 is comprised of several elements. The first element is physical element, network server 6. Network server 6 connects to network 40, and is itself connected to data storage device 4. The other elements illustrated in FIG. 1 for data center 80 (as well as clinic 10 and patient 50), comprise software components which are available in a web browser environment, of which is well-known to those skilled in the art. As such, they can be generally referred to as features or functions available to doctors, physician extenders, nurses and system administrators (will be described in greater detailed below) within data center 80 and clinic 10 on various intelligent devices, i.e., personal computers. Network hardware and services, including, but not limited to, routers, firewalls, LDAP services and related functionality associated with secure and robust networks is not shown, but, as one skilled in the art understands, are included in the embodiments of the invention.

The first function at data center 80 is system administration function 8. System administration function 8 allows authorized users to perform administrative and maintenance work to electronic care health management system (ECHMS) 100. Additionally, rule engine and business logic function 2 is included at data center 80. Rule engine and business logic 2 is not a user interface, per se, but a set of rules that incorporates the decision making abilities of ECHMS 100. It is in rules engine and business logic (rules engine) 2 that patient information is reviewed and recommendations are created according to highly advanced and sophisticated programs that incorporate the latest medical knowledge, as well as peripheral information such as insurance requirements.

Clinic 10 represents a CHF facility in which patients will visit to see physicians extenders and/or doctors and/or nurses, and to receive treatment in the form of medication, evaluation, and/or therapy. Clinic 10 comprises local clinic administration function 20, outcomes function 18, voice annotation function 16 (which may also require the use of an external physical device, not shown), clinical patient record (CPR) function 14, manual data entry function 12 (which may itself be comprised of a keyboard, for example), routine office tools function 22, alerts function 24, report support functions 26, medications 28, and recommendations functions 30. All the aforementioned functions will be described in greater detail below.

Patient 50, as illustrated in FIG. 1, generally embodies a patient at a remote location. For example, a patient may utilize a laptop, a personal digital assistant, or even a cellular phone capable of communicating over network 40 to interact with ECHMS 100. However, in the preferred embodiment, patient 50, as shown in FIG. 1, encompasses a patient physically in their house and having an intelligent device 52 connected to network 40, and having remote monitoring functions 54 and education and support function 56.

Although the majority of the discussion regarding electronic care health management system (ECHMS) 100 is specifically related to interactions between clinic 10 and patient 50, it is to be understood that clinic 10 and/or patient 50 might have the opportunity and benefit from interactions through network 40 to other information systems 60. These other information systems 60 might comprise pharmacies 62 (for electronic transfer of medication and prescription information), hospital 64 (for gathering of hospital records and/or transmittal of medical records from clinic 10), laboratories and diagnostic services 66 (for making of appointments, the transfer of medical records and/or reports between the laboratory and diagnostic services 66 an clinic 10), various federal agencies and/or departments 68 (which might comprise Health and Human Service Organization, Center for Disease Control, possibly even Medicare or Medicaid Departments), and of course, state and local agencies and departments 70 (which might comprise local healthcare facilities run by the city, testing and public safety institutions).

The following brief description describes the present invention generally. There are four main participants in the present invention of ECHMS 100, and each participates in different, though sometimes in an overlapping, manner.

1. Nurse Practitioner/Physician Extenders—The ECHMS 100 helps the nurse or physician assistant (PA) assist more patients by providing a clinical patient record keeping system that reduces workload and paperwork. The ECHMS 100 is browser-based, and allows the nurse or PA to:
Electronically monitor patients and set parameters for alerts.
Schedule patient appointments and lab tests.
Prescribe or recommend medications and treatment based on protocols.

2. Physician—The ECHMS 100 helps the physician assist more patients, more accurately apply evidence based medicine, and keep up with changing protocols for medications. The ECHMS 100 provides the physician with:
Flexible, rules based engines for developing treatment plans.
Scheduling, patient monitoring and assessment tools to reduce paperwork and overall workload.
Administration tools for generating reports and reviewing treatment plans implemented by nurse practitioners.

3. Healthcare Administrator—The ECHMS 100 helps healthcare administrators effectively run their disease management system. ECHMS 100 has a browser-based interface which allows administrators to:
Create and run reports on treatment outcomes.
View scheduling, workload, and prescriptions at a clinic level.

4. Patient—The ECHMS 100 will help patients monitor their treatment and provide feedback to the physician. ECHMS 100 will provide a browser-based interface allowing patients to:
Review or explore educational material on their condition.
Enter monitoring and assessment information and receive feedback on their progress.
Access their caregivers.

ECHMS 100 is comprised of the following major components:
Physician Extender/Physician Interface—Browser-based interface where the Physician Extender or doctor can control patient records, monitor patients, create patient plans, and manage scheduling.
Administrator Interface—Browser-based interface that allows system and protocol administration.
Patient Interface—Browser-based interface that provides patients with feedback on their progress, medication information, and general help on controlling their illness.
Patient Monitoring Devices—Electronic devices that collect and transmit data to the patient's medical record. The monitored records can be set to provide alerts to medical personnel if the patient's data (for example, weight) exceeds set thresholds. These patient monitoring devices may include a browser enabled device capable of communicating through remote monitoring function 54, intelligent device 52 and ultimately network 40 to clinic 10 and/or data center 80.
Database—A database that contains patient records, and allows for the creation of reports that support data mining and clinical research.
Clinical Patient Records—Electronic records that can be created and maintained in the system, and provide the immediate benefits of automation without the difficulty of converting entire existing medical records.

In addition, ECHMS 100 has the following features: content feeds (education, information (i.e., new therapies), reminders), E-mail, MD Home pages, and connection to local care services i.e. pharmacies, labs, equipment suppliers. Table I, shown below, details the features of ECHMS 100 according to an embodiment of the present invention, and the components of ECHMS 100 that supports those features. For example, patient monitoring and assessment is a feature of ECHMS 100. To provide that “feature”, ECHMS 100 has implemented an integrated wireless device for patients to use to monitor their vital personal statistics. In addition to the “patient monitor” component of ECHMS 100, there is an alert system. Certain participants will have access to some of these components, and some participants will have access to all. Each component will be discussed in detail below. Note that the “clinical patient record” (CPR) record does not appear in any one feature; instead, it is shared by “Patient Monitoring and Assessment” and “Patient Management”. Obviously, data that exists in the CPR must be updated when alerting patients, or when their wireless devices provide new readings, and the data is useful for managing patient care. These relationships will be described in detail below.

TABLE I

| FEATURE | COMPONENT OF ECHMS |
|---|---|
| PATIENT MONITORING | Remote monitoring |
| AND ASSESSMENT | (Wireless Device Integration) |
| | Subjective (How do you feel?) |
| | Objective (Actual Measurements) |
| | Education and Support |
| | (Alert System) |
| | Clinical Patient Record |
| | Manual Data Entry |
| PATIENT MANAGEMENT | Recommendation |
| | Rules Engine and Business Logic |
| | Routine |
| | Office Tools |
| | Call Logs |
| | To Do Lists |
| | Voice Annotations |
| | Calendar/Scheduling |
| | Bulletin Boards |
| | E-faxes |
| | Alerts |
| CLINIC AUTOMATION | Local Clinic Administration |
| | Elect. Prescription Pad |
| | Medication Reference Library |
| | Lab Notification |
| | Pharmacy Notification |
| | Electronic Signature System |
| | Clinical Patient Record |
| SUPER AND LOCAL SYSTEM | Report Support |
| ADMINISTRATION | Outcomes |
| | System Administration |
| | Reports |
| | Clinic Administration |
| | Audit/Log System |
| | Billing |

As described above, there are four main participants to the method and system of the present invention. The manner in which they participate, as well as several others will now be described in further detail.

Nurse. The nurse or physicians assistant (PA) will be the primary user of ECHMS 100. Nurses are well educated and usually computer savvy, and use established and time tested procedures or workflows. Changes to those time-tested procedures will be resisted unless there is sufficient motivation to do so (that is, a good return on their investment of time which).

Nurses' job functions in ECHMS 100 may include: New Patient Enrollment; Create/Modify Patient Records; Schedule Visits; View/Set Appointment Reminders; Print Records; Generate; Treatment Plan (Using Rules Engine); Approve or Authorize Protocol Outcomes (treatment and schedules); View/Modify Patient Monitoring/Alert Levels; Efax/Print Prescriptions; Efax/Print/Mail Information to Referring Physician; Efax/Print/Mail medication schedules, information and other educational materials to patients.

When operating within ECHMS 100, nurses will have the need to know certain information, and thus access specific elements of the ECHMS 100. These include: Shared Calendars (Edit privileges); Patient Records (Edit privileges); Rules Engine (User); Report Generation Engine (User). The comments within the parentheses indicates the type of access nurses are allowed to the aforementioned elements of the ECHMS.

It is expected the nurses who utilize ECHMS 100 will have a “Medium” computer skill level and a medium-to-high medical education level.

Program Doctor. The program doctor will have all physician extender functions, as well as approval of initial patient plan and the ability to view all system level reports. The program doctor must also be able to supervise the physician extenders, and query how treatments have been implemented. For this reason, the doctor must be able to view a history of the day's work, and must also be able to send questions to the physician extender in an electronic format that is acceptable to the current Health Insurance Portability and Accountability Act (HIPAA) and clinic regulations. The program Doctor may be the clinic administration, but typically is an administrator, or a doctor who has same greater level of responsibility at clinic 10 for implementation of ECIMS 100. The program doctor, is, in effect a medial doctor with managerial responsibilities.

Physician. Physicians' job functions in the ECHMS 100 may include: New Patient Enrollment; Create/Modify Patient Records; Schedule Visits; View/Set Appointment Reminders; Print Records; Generate Treatment Plan (Using Rules Engine); Approve or Authorize Protocol Outcomes (treatment and schedules); View/Modify Patient Monitoring/Alert Levels; Efax/Print Prescriptions; “E-prescriptions” if available; Efax/Print/Mail Information to Referring Physician; Approval of Initial Patient Plan; and View all System and Program Administration Reports.

As with the nurses, physicians will have the need to know certain information, and thus must be able to access specific elements of ECHMS 100. These include: Shared Calendars (Edit privileges); Patient Records (Edit privileges); Rules Engine (User); Report Generation Engine (User); Plan Sign Off.

Physicians should exhibit a “Medium” skill level in regards to computer usage, and of course, high medical education level.

Secretary. The secretary's responsibilities are a subset of the nurse practitioner. That is, the secretary will be primarily responsible for administrative tasks such as appointment scheduling, maintaining the calendar, and some patient record maintenance. However, the secretary will not have privileges for creating treatment plans or prescribing medications.

The secretary's job functions may include: New Patient Enrollment; Create/Modify Patient Records; Schedule Visits; View/Set Appointment Reminders, Billing; and Print Records.

The secretary's information requirements are fairly limited. There may include: Shared Calendars (Edit privileges); and Patient Records (Edit privileges). Generally speaking, the required computer skill level for secretarial positions is basic, and medical education is generally not required.

Program Director. The program director manages the CHF clinic. This person could be a program doctor, with the additional program director rights and responsibilities. The job functions of the program director may include: Management of the CHF clinic; Coordinating the program doctors to determine and modify medical protocols as needed; Working with System Administrator on high-level decision-making issues to implement specific processes and protocols; Granting access rights to system users; and Running reports

The Program Director has the highest classification of access to the ECHMS 100 functions: This is referred to as “Super User” access. Program Directors will need to exhibit “Medium” computer skill levels and, preferably, a “high” medical education level.

System Administrator. The system administrator typically acts on the orders of the program director to set-up, maintain, and administer ECHMS 100. The system administrator's job functions may include: Adding Users to the System; Setting or Modifying Access Privileges; Administering System Logs; Creating and Executing a Backup Process; Auditing System Performance; and Acting primary contact for all Technical and Licensing Issues.

The system administrator's job functions may encompass a wide range: from setting up a new clinic to editing medical protocols. The System administrator manages the information capabilities the software provider as a service to healthcare delivery systems.

Job Functions. May include: Implement and Test Protocols (in conjunction with System Medical Director); Edit System Parameters (for example, backup schedule); Set up New Clinic—Create, Modify, or Delete New Clinic Instance; Data Mining & Reporting; Export Data (for example, dump data back to clinics as part of a “Clinic Closing Procedure”); Device Setup; Training; Help Desk; Customer Service.

The system administrator will enjoy SuperUser Access to all clinic System Functions, and must have a “high” computer skill level, and no, or low medical education.

Program Administrator. The program administrator has no input functions, but typically runs reports on a wide range of system information. The program administrator is located within a health enterprise. The program administrator's job functions may include: running reports on program metrics including: number of patients in program; number of visits per time period; average length of stay; telephone call volume; number of hospitalizations; and basic insurance reimbursement types; staff performance and productivity.

The program administrator will have access only to the report generation engine, at an administrative level. The program administrator's computer skill level need only be “Medium” but medical education level should be “high”.

FIG. 2 illustrates a flow diagram of a method for enrolling a new patient and for entering new patient information into the electric care health management system in accordance with an embodiment of the invention. The system and method of the present invention encompasses at least eight separate processes, in which particular actions occur in assisting to provide the desired result of an electronic system and method of healthcare management. Each separate process has preferred participants, and is accompanied by a figure which assists in understanding the nature of the process with reference to the present invention.

The first process is the referral and enrollment process (illustrated in FIG. 2). This process encompasses the enrollment of a new patient into ECHMS 100. Users of this process are primarily the secretary, but anyone with user rights can perform the use cases contained in this scenario. Through use of referral and enrollment process 200, patient data is entered into ECHMS 100, where it can be accessed when the patient visits the clinic, or during telephone consultations.

Several use cases are contained in this scenario (a “use case” is an example of how the particular process is used, i.e., what function it performs). The first may be referred to as log external referral use case 210. In log external referral use case 210, the user enters referral information into the patient record. The next use case is obtain general information use case 220. In this use case, the user enters general patient information into the patient record and schedules appointments. The last use case is first enrollment visit use case 240. In this use case, the user completes the patient CHF electronic record and enters the patient into the system. Each use case in referral and enrollment process 200 will now be discussed in detail.

Log external referral use case 210 is used to enter external referral information into a patient's electronic record. In this instance, this data may not be collected at the same time as other information. Log external referral use case 210 begins with step 212. In step 212 external referral information will be part of the patient's electronic record, but may not be collected at the same time as other patient information.

The user selects “Add Patient” from the navigation bar. A blank patient record appears. Then, in step 214, the user enters referral information (if any). This may include: name; contact information; referring party data; disease state (for example, CHF; diabetes; respiratory) and the reason for referral.

For example, for a referral exhibiting or having CHF, the following are reasons for the referral: ischemic heart disease; anti-coagulation; diabetes; respiratory; medication support; case management; or other.

The second use case illustrated in FIG. 2 is obtain general information use case 220. Obtain general information use case 220 is used to collect general patient information before the patient actually visits the clinic. If this is the case, after patient data is entered, the user may want to schedule an appointment for the patient.

Obtain general information use case 220 begins with step 222. In step 222 the user will enter general information about the patient. This will include date-of-birth; gender; ethnicity (optional); other medial providers; whether or not the patient is in outpatient/inpatient status (and if so, what hospital and room number); insurance information (policy number, contact information); and whether the patient wishes to schedule an appointment (Note: This is a separate Use Case).

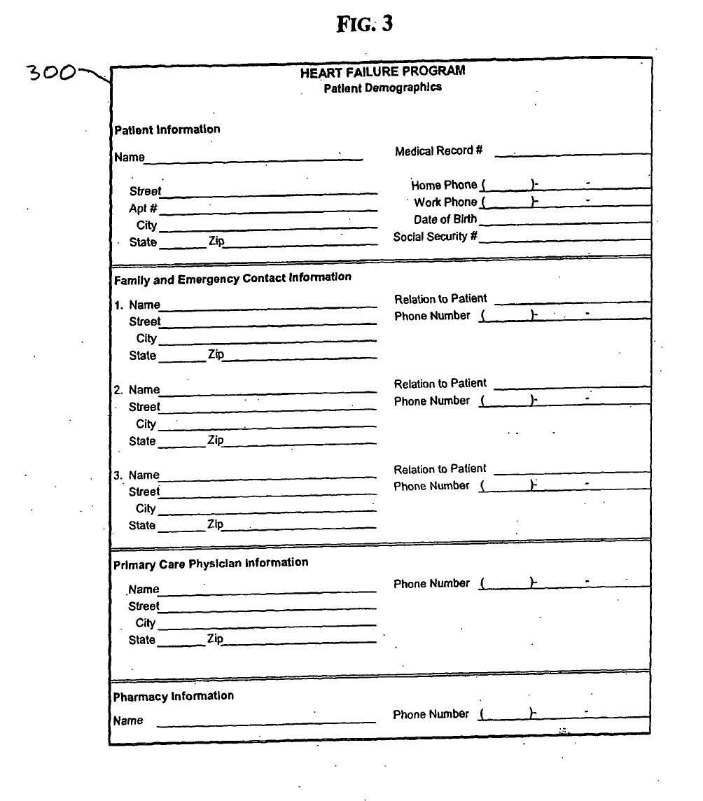

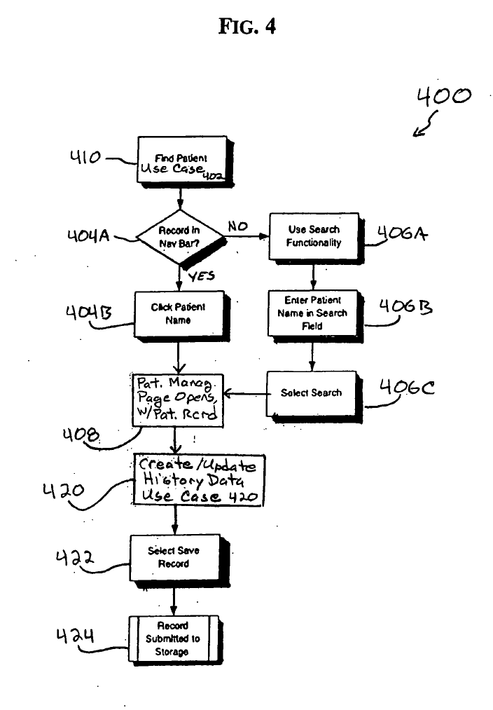

The final use case of referral and enrollment process 200 is first enrollment visit use case 240. First enrollment visit use case 240 completes the patient record. The patient completes “patient demographics” form 300 as illustrated in FIG. 3. First enrollment visit use case 240 begins with step 242, in which the user obtains enrollment information from the patient. This will include medical information releases and consent confirmation. Next, in step 244, the user will obtain the patient's goals. This is then followed by obtaining patient data (step 246), providing initial education to the patient (step 248) and reviewing the patient's goals (step 250). Finally, the user saves the patient record in step 252.

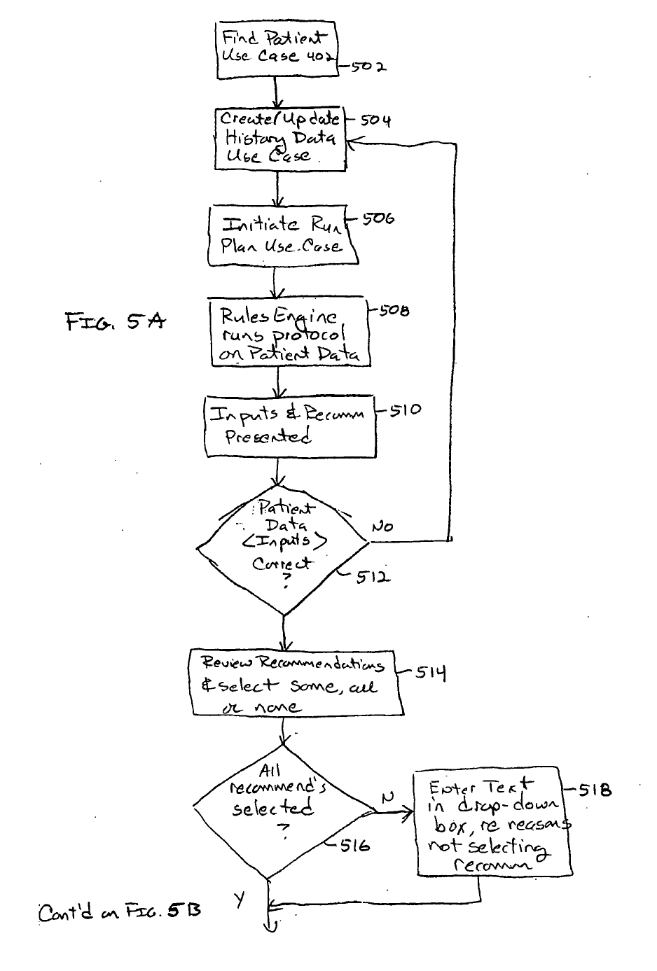

FIG. 4 illustrates a flow diagram of a method for creating and updating a patient record in an electronic care health management system in accordance with an embodiment of the present invention. The second process of the present invention, create/update patient record process 400, involves adding new information to an existing patient record. The primary user of this process will be the physician extender, which may be a healthcare provider other than the physician, but could also be the nurse or secretary entering data on behalf of the doctor or physician extender. Only the doctor (and in some cases the physician extender) will have access to create plans and prescribe medications. Create/Update Patient Record process 400 ensures that patient information is accurately entered into the clinical patient record. As a pre-condition for use of this process, the patient must be enrolled, and the user must have access rights and be logged on.

The following use cases are utilized in create/update patient record process 400: Find patient use case 402, in which the user finds the patient record, and create/update history data use case 420, in which the user enters patient information. Each will be discussed in detail.

In reference to the first use case, find patient use case 402, there are two methods; the first is shown as steps 404A, 404B and 408, and is the simple case. In this method of finding a patient record, the user will attempt to locate the patient on a navigation bar (shown as decision step 404A). If the patient is part of a scheduled event for the day (i.e., an the calendar or “to do” list) the patient name should appear in the list of patients contained in the navigation bar (“Yes” path from step 404A). If the patient's name does not appear in this list (“No” path from decision step 404A), then the user must enter all or part of the patient's name in the search field in the navigation bar, and select “find”. This constitutes the second method to find a patient record, and is comprised of steps 404A, 406A, 406B, 406C and 408. When the patient record is located by ECHMS 100 (through either method of finding a patient), the patient management page (step 408) opens populated with the patient data.

The second use case is create/update history data use case 420. Once the patient has been located and the patient management page is open, the record can be updated. The user will then select the relevant tab to view patient data. The tabs include: patient information; medications; labs; lifestyle; and appointments. Additional relevant patient information will include insurance and/or billing information. The user may then enter new, or modify existing data as required. The user will then select save record in step 422, and in step 424, the newly updated patient record is submitted to storage.

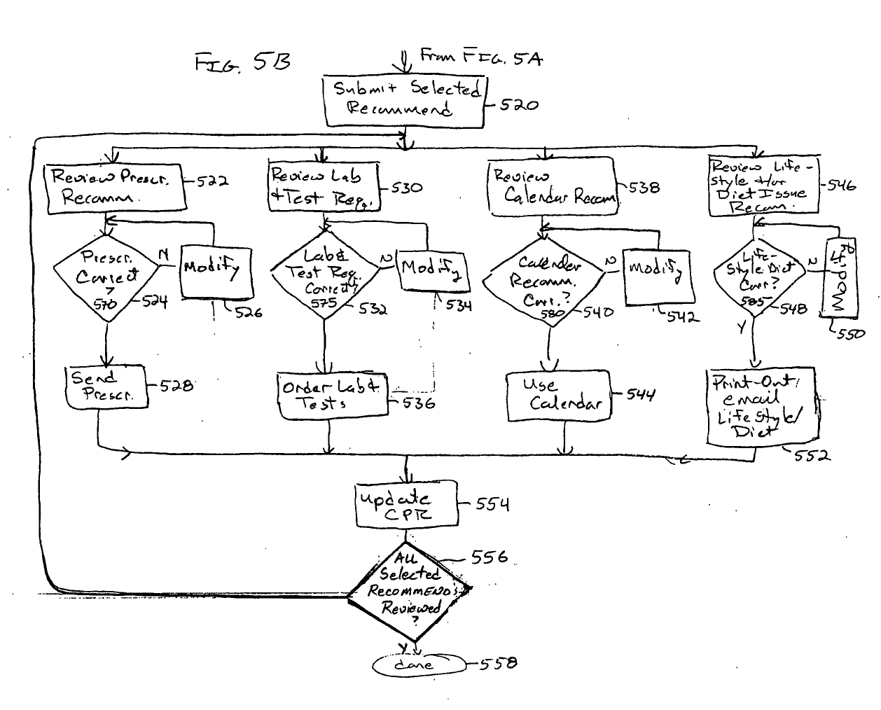

FIGS. 5A and 5B illustrate a flow diagram of a method for running a treatment plan in an electronic care health management system in accordance with an embodiment of the present invention. The third process of the present invention is run treatment plan 500, and this includes applying the medical rules engine 2 to a patient, approving or rejecting a recommendation, and issuing prescriptions and lab/test requisitions. Based on the patient data that has been entered into the system, the system applies the predetermined rules to get the recommended plan of action, and the user reviews the results.

Several use cases are involved in utilizing run treatment plan 500. These are: find patient record use case 410; create/update history data use case 420; run plan use case 560; submit plan use case 565; selecting medications use case 570; lab & test requisitions use case 575; calendar use case 580; and lifestyle & diet issues use case 585. Each will be discussed (unless already previously discussed) in detail as used in run treatment plan 500.

Users of run treatment plan 500 include the nurse, PA, program doctor and medical director. The desired outcome from utilizing run treatment plan 500 is a completed plan in place for the patient that includes medications, prescriptions, lab tests, follow-up visits, and lifestyle recommendations.

Several pre-conditions must exist prior to using run treatment plan 500. First, the patient must exist in the system. Second, patient information needs to be “sufficient” (i.e., enough data points for rules engine 2 to run). And third, the user must have rights to run the plan function, and write prescriptions. If physician extenders lack prescription rights to write prescriptions, ECHMS 100 provides an automated approval process.

Run treatment plan 500 begins with step 502, which utilizes find patient record use case 402, to find the patient record. Then the user, in step 504, utilizes create/update history data use case 420 to modify any vitals as needed under the appropriate tabs. The next step is step 506, in which the user initiates run plan use case 560. Step 508 shows the rules engine running the protocol on the patient data. In step 510, the rules engine returns recommendations and a list of the inputs used to generate the recommendation. The user, in step 512, will review the inputs to determine if they are correct (decision step 512). If any of the patient data is incorrect (“No” path from decision step 512), the user will be redirected to create/update history data use case 420, and will select the appropriate tab to change any faulty data (step 504). The user would then return to the plan page and select “initiate run plan use case” again (step 506). Presuming that at some point all the patient data is input correctly (“Yes” path from decision step 512), the user will be directed to review the recommendations (step 514), and check (accept) those that are appropriate (step 516). If the user decides that only some or none of the recommendations are appropriate, the user will be directed to enter text that explains the reason for rejecting the recommendations (step 518). For recommendations that were not checked, the user has rejected the plan and must explain why and indicate an alternate course of action (if any). Rather than processing the recommendation, the user has a free text box or drop down selection box to indicate the reasons for rejecting the plan and their recommendation.

Run treatment plan 500 continues to step 520. In step 520, the user will select submit, which utilizes submit plan use case 565. A pop-up window appears with the first recommendation that was checked in step 516, and displays the steps to process that recommendation. For recommendations that were checked, the user has accepted the plan and the steps in the pop-up window guide the user to generate prescriptions (select medications use case 570) requisition labs/tests (lab & test requisition use case 575), schedule follow-up visits (calendar use case 580), and print lifestyle information (lifestyle & diet issues use case 585).

An optional Wizard help program, which aids the user in following the correct steps in ordering prescriptions and/or lab tests, use of the calendar, and obtaining lifestyle and/or diet educational information may be present at any time during method 500, and is easily recalled by clicking on the wizard icon opening it. The Wizard help program might also pop-up whenever new information is entered.

Following step 520, in which the user has utilized submit plan use case 565, it is possible the user may have several recommendations to chose from when submit plan use case is utilized (i.e., the “submit” button is clicked), the ECHMS 100 returns one or more recommendations based on the patient data that was submitted. The recommendations may be viewed in any order; one, some or all of them may be present. Implicit after each recommendation is the option to view it again or a different one. Each recommendation “path” will be discussed in order, but, as one skilled in the art understands, the recommendation actions may be viewed in any order.

If the Rules Engine has recommend a prescription that may be the first recommendation the user selects, in step 522. If the rules engine recommends prescriptions, and the user concurs (“Yes” path from decision step 522), selecting medications use case 570 will run and present prescription information. The user then must review the prescription information, and determine if they are correct. This may include a class of medications and a target dose according to the rules engine. This is shown as decision step 524. The user will then select the brand or generic drug that meets the requirements of the patient (step 562). Alternatively, the user will have access to a third-party drug list (such as Medscape) to select the medications. According to the settings of the system, the user may have access to a set of brand and generic drugs that are part of the protocol and a set of drugs that are outside of the protocol. In this case, first the user tries to find a drug within the protocol, but can select “non-protocol” to expand the available selection. Alternatively, the user may have access only to the brand and generic drugs in the protocol (medical director setting control). In this case, the user selects the drug from the protocol list.

If the prescriptions are not correct (“No” path decision from step 524) the user will have an opportunity to modify the prescription information. Selecting medications use case 570 will be used in step 524 to modify the prescriptions if necessary. When the rules engine is directed to generate a prescription (“Yes” path decision from decision step 524), it will also perform a formulary check. A formulary check is a verification of insurance coverage for prescribed medications against the patient's insurance coverage. If what the rules engine prescribes is covered, or on the “accepted prescriptions” list of the patient's insurance plan or program, then there is no problem. If, however, the rules engine suggest a medication that is not on the insurance plan's “accepted prescriptions” list, the physician extender or doctor must seek an alternative medication that fulfills the patient's requirements and is on the “accepted prescriptions” list. These changes will be shown and implemented in steps 524 and 526. Additionally, selecting medications use case 570 may check for probable adverse medication events and alert the user about potential contraindications.

When all the prescription information is correct (“Yes” path decision from step 524), the prescription information will be sent to the appropriate facilities. The user will select the prescriptions tab, which is where all of the prescriptions have loaded up and are ready to be sent to the patient's pharmacy via dedicated data network, efax or printed for the patient to carry home. After prescriptions are sent and/or printed, the medications appear on the patient's meds tab (the CPR is updated). The next time treatment plan 500 is run, the new meds will be taken into account by the rules engine. The user can print an easy to read list of all the patient's medications, including a schedule of when to take each pill, for the patient to take home. After the prescription information has been settled, it is possible other actions are necessary. These are lab and test requisitions (step 530), calendar use (step 538) or lifestyle and/or diet issues (step 546). Each will be discussed in turn.

Lab and test requisitions use case 565 is a second recommendation that the rules engine might offer. When the rules engine recommends a lab or test, the physician extender must send a requisition to the lab informing that the patient will be coming in for a certain procedure (lab or test). The requisition goes by fax to the facility (which can be a drop-down feature on the program page. This feature allows text message entry, contains frequently used fax numbers (by name and number), and allows manual fax number entry). The requisition contains a fax back number so that the results will go to the clinic and can be attached electronically to the patient file. Other means of electronic communication may be used in addition to facsimile, such as email, EDI and so on.

If a lab and test recommendation has been presented, the user may select it as step 530. Then, lab and test requisitions use case 575 is run, and all the appropriate lab and test requisitions forms are generated and presented to the user. In step 532, the user must decide if those lab and test requisitions are warranted or complete. If not (“No” path from decision step 532), the rules engine and lab and test requisitions use case 575 will generate a different set of lab and test requisition, based on the new information the user has included, or, which lab and test requisitions have been canceled or modified. Or, optionally, the user may simply select and/or modify the lab and tests to suit the user's desires. Once all the lab and test requisitions are correct, the user accepts them (“Yes” path from decision step 532) and the next step is step 536.

In step 536, rules engine 2 orders the lab and tests. Again, a “formulary” check may be performed, in that the insurance company has agreements with certain diagnostic and test facilities. The requisitions are forwarded, reminders/directions (if necessary) are printed out, and the user may then have the option of selecting another recommendation, if necessary.

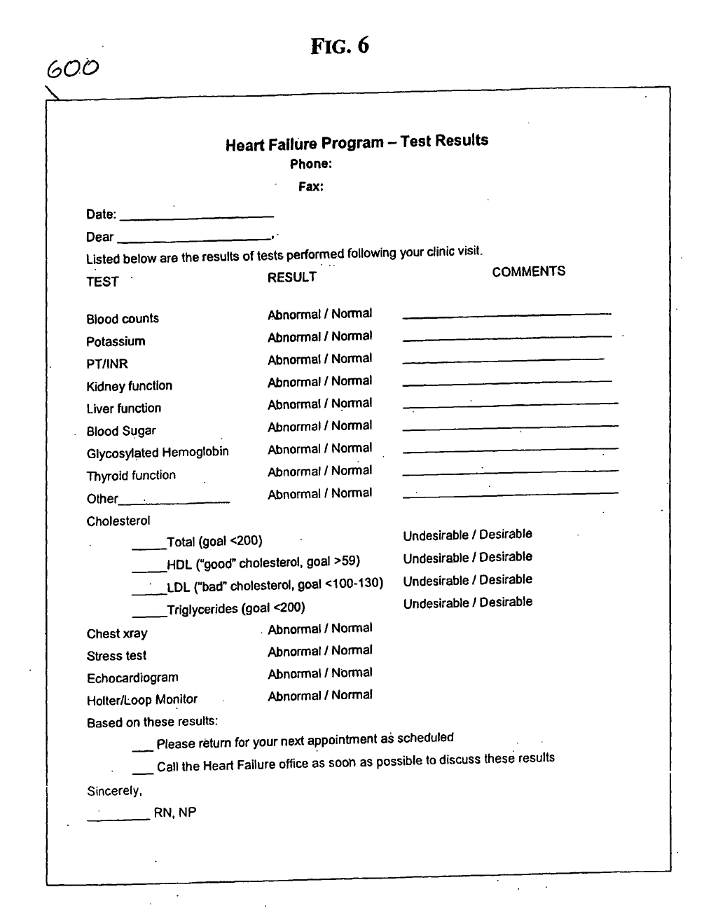

The physician extender or secretary must transcribe the results into the labs tab if results are to be part of the record and interpreted by the rules engine. The physician extender or secretary must transcribe lab results in the Labs tab to generate a summary of results that can be printed and mailed to the patient. This may be accomplished by several different methods. One method is to enter the data manually, i.e., by actual keyboard entry. Another method could be completely digital and automatic, where the retained lab test results are in a digital format recognized by the HCMS. The third method might be a combination of the two, where the data is encoded, but must be manually retrieved. For example, a bar-code, magnetic strip, tape, optical disk, floppy etc., are all examples of this last case. See, for example, FIG. 6 which illustrates a heart failure program test results data form 600, used in accordance with an embodiment of the present invention. The E-Care health management system according to the present invention may also print a label or envelop to accompany the form illustrated in FIG. 6.

Calendar use case 570 may also be used in run treatment plan 500. Rules engine 2 might also determine that additional appointments are needed for proper care of the patient, or whether the patient desires additional appointments. Through use of calendar use case 570, the user can access the calendar to schedule recommended follow-up visits. The user can print a schedule of all upcoming office, lab and/or test appointments for the patient to carry home.

The user selects review calendar recommendations in step 538. Rules engine 2 has generated appointments based on protocols for visits for the particular condition/affiliation of the patient. These appointments are checked against the dates of lab tests (which are shown in the calendar print out) and also the workload of the doctor the patient is working with. In addition, vacation schedules, holidays known patient preferences and possibly other factors may be taken into account.

If the calendar recommendations are not correct (“No” path from decision step 540). The user will continue to make changes until the calendar is okay (“Yes” path from decision step 540); then, method proceeds to step 544 and the calendar is printed out (and/or e-mailed/faxed to a destination of the patients' preference) for the patient to take home. Following step 544, the user has the option to view another recommendation (“Yes” path from a decision step 556), and the user can again select any of the recommendations originally presented. Recommendations that have already been reviewed have an indicator showing. The last recommendation to discuss is lifestyle and/or diet issue recommendations.

Lifestyle and diet information, is the last recommendation to be discussed that rules engine 2 might propose as part of its recommendations. These attempt to help the patient help herself, by offering pro-active steps to take to alleviate conditions or symptoms of the illness. If a recommendation is a lifestyle or diet issue, the user may print out information for the patient, or indicate that information has been communicated verbally or in each clinic's pre-printed brochure.

Following step 520, the user may select the lifestyle and/or diet issue recommendation, if any, presented by rules engine 2. The lifestyles and/or diet issues are presented in step 546, and in step 548, the user reviews the recommendations and decides whether or not they are proper. If the lifestyle and/or diet issue recommendations are not proper (“No” path from decision step 548), the user may modify them (step 550) and again review for completeness and/or accuracy. Once the lifestyles and/or diet issues recommendations are accepted (“Yes” path from decision step 548), a printout is made in step 552, with the option of e-mailing/faxing to the destination of choice of the patient.

Following each review of a recommendation, the clinical patient record is updated (step 554), and the rules engine asks whether any other recommendations are to be reviewed. If yes, the method returns to steps 522, 530, 538 and 546, by presenting each recommendation again (“Yes” path from decision step 556). If no, (“No” path from decision step 556). Additionally, because the CPR for the patient contains insurance and/or billing information, when the patient's CPR is updated, a claim form and/or a bill will be generated. This occurs as part of step 554. The user is done with recommendation review, and treatment plan 500 is complete.

Execution of the treatment plan, as previously described, generates an audit trail of billable services that may be manually or automatically parsed to generate a paper or electronic claim. This process may be supervised by the secretary or another professional (billing specialist, accountant, program doctor, program director, among others) on the care team.

One skilled in the art recognizes that following the review of each selected recommendation the clinical patient record need not be updated; the clinical patient record may be updated only after all the selected recommendations have been reviewed.

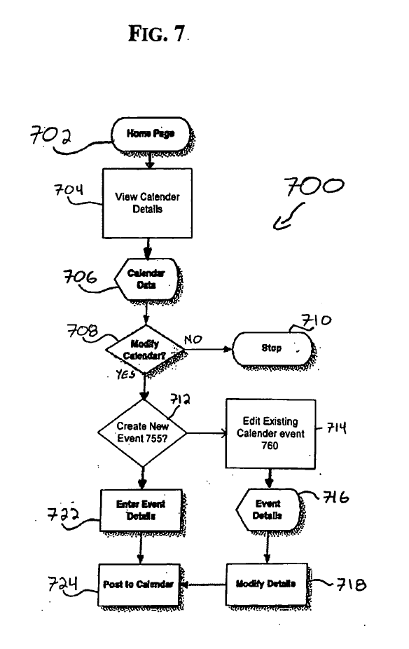

FIG. 7 illustrates a flow diagram of a method for calendar and event scheduling used in an electronic care health management system in accordance with an embodiment of the present invention. The fourth process of the ECHMS 100 is calendar/event scheduling process 700 which is a central system that manages all of the appointments and tasks created by users for themselves, the clinic and patients. Calendar/event scheduling process 700 allows double booking and provides typical office appointment management needs. The rules engine also sets appointments on the calendar. Calendar/event scheduling process 700 is compatible with many handheld personal digital assistant devices.

Users of calendar/event scheduling process 700 will primarily be the secretary and physician extender, though anyone (such as the program doctor) with access can set a schedule and may have permission to affect patient and clinic calendars. Certain pre-conditions are necessary prior to use of calendar/event scheduling process 700. For example, the user must have rights to enter the system and access/modify calendars.

The calendar is the first page a user sees after logging onto the system. There are four view options to choose from: My view (a view of the schedule for any given day, week, month for that particular person); Clinic view (a view the schedule for any given day, week, month for the clinic as a whole); Doctor view provides the user access to views of the doctor(s) calendars in the clinic); and All view (which overlays all of the views so the user can see availability and conflicts). There is also an appointments tab within the patient record that is linked to the calendar system. Each of the views will now be explained in greater detail.

My View. The my view option shows all of the appointments, on a given day, for the individual who is logged on. Appointments include patient visits, meetings, vacations, etc., each day also has a “to do” list associated with it. The “to do” list is populated in one of two ways: the user enters free text items or a scheduled lab or test has automatically placed a follow-up reminder on the user's schedule.

Clinic View. The clinic view option shows all of the appointments in the clinic as a whole. Depending on the work habits in a clinic, the physician extenders may all operate off the clinic view for patients, and use my view just for vacations, and individual meetings. If a patient were assigned to a specific physician extender, then that appointment would appear in the physician extenders my view for that day.

The “to do” list might also be shared in the clinic view, depending on how the clinic is set up. If the physician extenders share in making all follow-up calls, then they will move down the joint list, checking items off as they are done. If the physician extenders do not share “to do” lists, then each item appears on my view. It is assumed that the physician extenders will have read-write access to each other's calendars (clinic view only), so if someone is unable to take care of their own “to do” list, a colleague could access it and take care of it. Each physician extender can still have a “to do” list in my view where items that are not directly related to a patient can be entered. Any incomplete items carry from one day to the next until they are removed from the list.

Doctor View. The Doctor view option shows when the doctor(s) affiliated with the clinic have scheduled the dates when they will be in the clinic, on vacation, etc. This allows the physician extenders to know when the doctors are available and who is supervising on any given day.

The following use cases are utilized in this scenario: View calendar details use case 750 (this allows the user to navigate to and view details for a particular event); Create new calendar event use case 755 (this allows the user to create a new event); and Edit existing calendar event use case 760 (this allows the user to open a calendar event for editing). Each of these use cases will now be explained in detail, with respect to calendar/event scheduling process 700, as shown in FIG. 7.

Calendar/event scheduling process 700 begins with the user at the home page, in step 702. There, the user will select view calendar details use case 750 in step 704. There may be up to four calendars accessible through the physician extender home page. These calendars are my view doctor view, clinic view and all view. Each calendar can be viewed in either a daily, weekly, or monthly view basis. After selecting which calendar, the user will select a calendar view. The options are: today; week; and month. Following selection of the calendar view, the user will locate the event of interest by scrolling the selected calendar view until what he desires appears.

In step 706 of calendar/event scheduling process 700, calendar data is displayed. In decision step 708, the user must decide whether to modify the calendar: If no, then the user is finished with calendar/event scheduling process 700 (“No” path from decision step 708). If the user does desire to modify the calendar, the user proceeds to step 712, and decides whether to create a new event. If yes, then the user has selected create new calendar event use case 755.

Within create new calendar event use case 755 there are two methods for creating calendar events. First, the user can select a time in the calendar that is not occupied by another event, which opens a create event window. Second, the user can select the modify existing button. Calendar events must, of course, be time specific. Events that do not have an associated time are entered in the “to do” list. Therefore, the user will either select an empty time slot in the calendar component, or select “to enter a new event”. If the former, the user will enter event details in the aforementioned time slot. Or, if the latter, the user will then populate the new event window. These steps are shown collectively as step 722. Finally, the user will post the new information to the calendar (step 724).

The no path from decision step 712 means that the user wishes to modify an existing event. To do this, the user will utilize edit existing calendar event use case 760, shown in step 714. Here, calendar events can be edited by selecting them in the calendar component. Search functionality will also allow users to quickly navigate a large calendar. The steps involved in exercising edit existing calendar event use case 760 begin with navigating to a calendar event. The user will then select the desired event. Following this, an edit event window appears. This is shown as step 716 in FIG. 7. Then, in step 718, the user may modify data as required, and select submit. Thus, as discussed above, the calendar will have posted to it the changes entered by the user via steps 714-718.

The following examples illustrate ways in which the calendar may be used. First, a patient calls to make an appointment. The secretary opens the clinic calendar and selects a time for the patient according to the patient's availability and the clinic openings. The secretary types in the patient's last name, and the system automatically identifies the file, linking the date and time with the record. The appointment time loads into the patient's calendar on the appointment tab. If the patient is new, the secretary selects, “add new patient”, enters the name and contact information for the patient and sets the appointment (refer to first enrollment use case 140, discussed above). The patient's name appears on the clinic calendar for that date. If the clinic assigns physician extenders to patients (rather allowing the physicians to elect their patients), the secretary assigns the patient to a physician extender. The patient's name will now appear on that physician extender's schedule as well. When the date arrives for the patient's appointment, the system sends the link to the file to the left navigation bar so the physician extenders can access all of their patient records, listed in alphabetical order, for the day.

As a second example, suppose the physician extender has just seen the patient and concluded that the patient needs a lab, a test and a follow-up appointment in two months. The lab and test require requisitions will be sent to the appropriate facilities. The physician extender asks the patient what day to schedule the test, and sends requisitions that reflect that date. That date is entered automatically on the patient's appointments tab. The physician extender is prompted for a reminder to check on the labs and tests in n days, and the reminder posts to the “to do” list for the day the physician extender decides to make the follow up calls. The physician extender checks the clinic schedule to set a follow-up appointment for the patient; this date also appears on the patient's appointment tab. The physician extender can print the appointment tab for the patient to carry home. Anytime the physician extender wants to know what the patient has scheduled, they can go into the patient record and select the appointment tab.

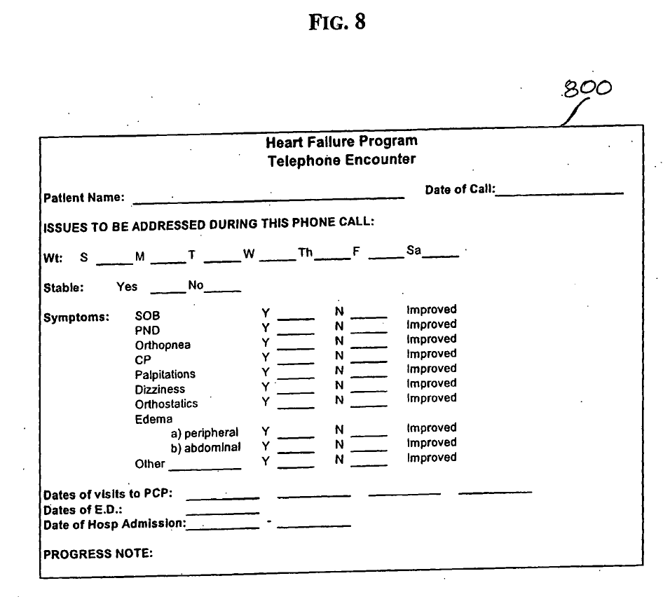

FIG. 8 illustrates a telephone encounter form used in an electronic care health management system in accordance with an embodiment of the present invention. The telephone encounter form is a separate data entry field within the “patient history” window where the physician extender records information about encounters with, and calls to, patients.

Users of telephone encounter form 800 may be performed by either the physician extender or secretary. The result of using telephone encounter form 800 will be accurate entry of contact data, and a consistent record process that will help the clinic bill for time spent on calls. As a pre-condition for use of telephone encounter form 800, the patient record must exist and the user must be logged on.

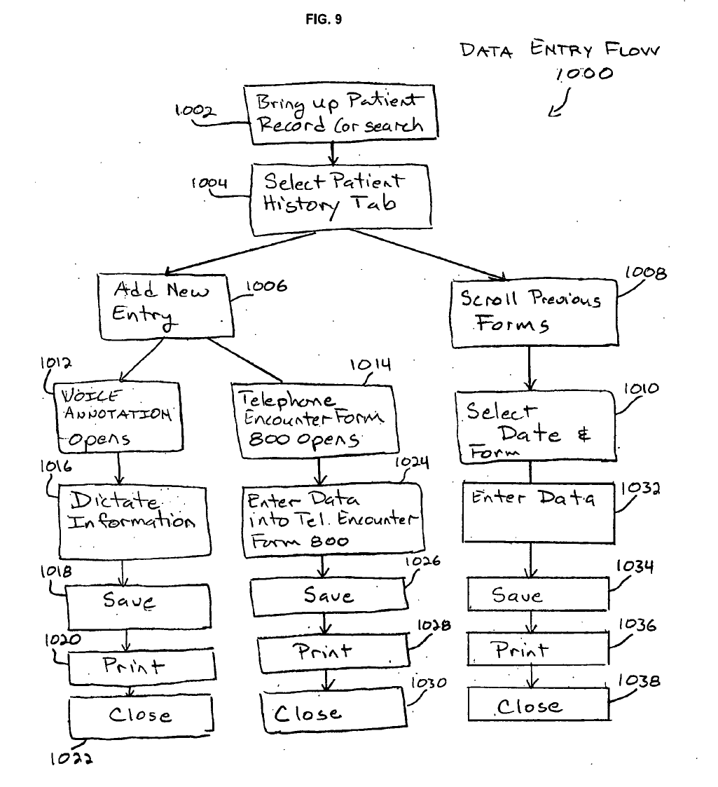

FIG. 9 illustrates a flow diagram of a method for entering patient encounter data into a clinic patient record used by an electronic care health management system in accordance with an embodiment of the present invention. Use of voice annotation device and telephone encounter form 800 is shown in encounter data entry flow diagram 1000. First, the user will bring up the patient record in step 1002, and select the Patient History tab (step 1004). Then, the user then will select either add new entry, step 1006 (to enter data regarding a new encounter or new telephone call), or may scroll by reverse chronological order to retrieve forms from previous interactions, step 1008, to edit data from previously entered forms. If the latter is chosen, then by clicking on any date (step 1008), the user opens up the encounter form that was completed on that date. The “add new entry” command (step 1006) prompts the user to select either voice annotation (step 1012) or telephone encounter form 800 (step 1014).

If the user has selected voice annotation in step 1012, the user sees a window that describes how to use the voice annotation device. The user simply follows the directions and speaks the information desired to be recorded. It is digitized, saved, and transcribed. This then becomes another digital record that can be manipulated, saved, and forwarded at will. It can even be printed out if necessary. The user selects save (step 1018) and the voice annotation is entered into the system and may also be optionally printed in triplicate and submitted to billing (step 1020), added to the paper file, etc. The user can flip to any tab before, while and after entering information in the log sheet. The user then closes the voice annotation window in step 1022.

If the user has selected telephone encounter form 800 in step 1014, the user sees an online version of the paper telephone encounter form that is used in the clinic top document all phone calls that are received by the clinic. Telephone encounter form 800 automatically appears, and guides the user through the call. Fields are pre-populated with data from other tabs as much as possible. The user fills in the appropriate fields during and after the call (step 1024), and selects save (step 1026) when finished. The user can flip to any tab before, while and after entering information in the log sheet. The new entry is filed by date in the patient record, along with all of the other forms that have been filled out for that patient. Telephone encounter form 800 may be optionally printed in step 1028, and saved in step 1030.

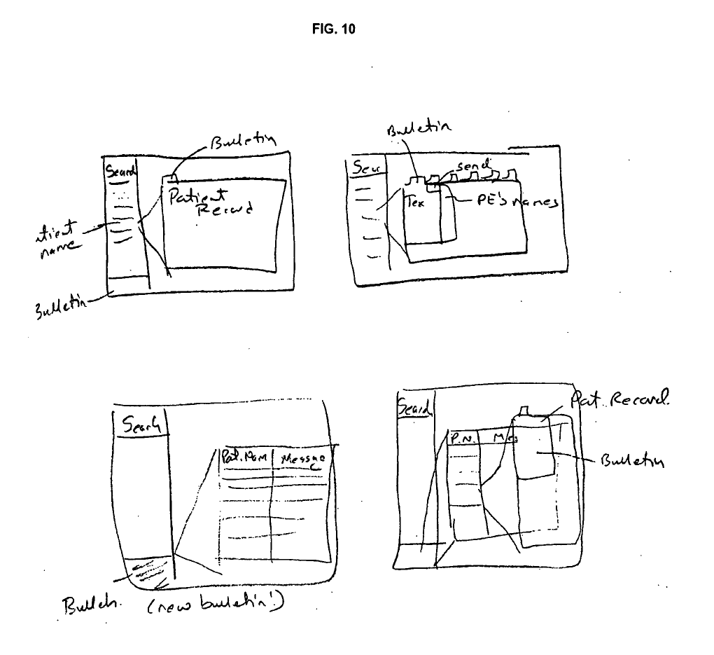

FIG. 10 illustrates a doctor/physician extender bulletin board display interface for use in an electronic care health management system in accordance with an embodiment of the present invention. Doctor/physician extender bulletin board comprises a bulletin board 1100 in which the doctor can direct questions to the physician extender regarding a specific patient. The log resides within the secure system and does not violate HIPAA rules by using email. Under certain circumstances it is permissible to discuss patient's diagnosis and potential treatments through use of e-mail. Users of the doctor/physician extender bulletin board 1100 would be the clinic's Doctors, physician extenders and possibly selected secretaries. By using doctor/physician extender bulletin board 1100 the Doctors can review a patient's treatment record and convey any questions or comments to the physician extender. As a pre-condition to use, the users must be authorized to access doctor/physician extender bulletin board 1100 and be logged onto the system.

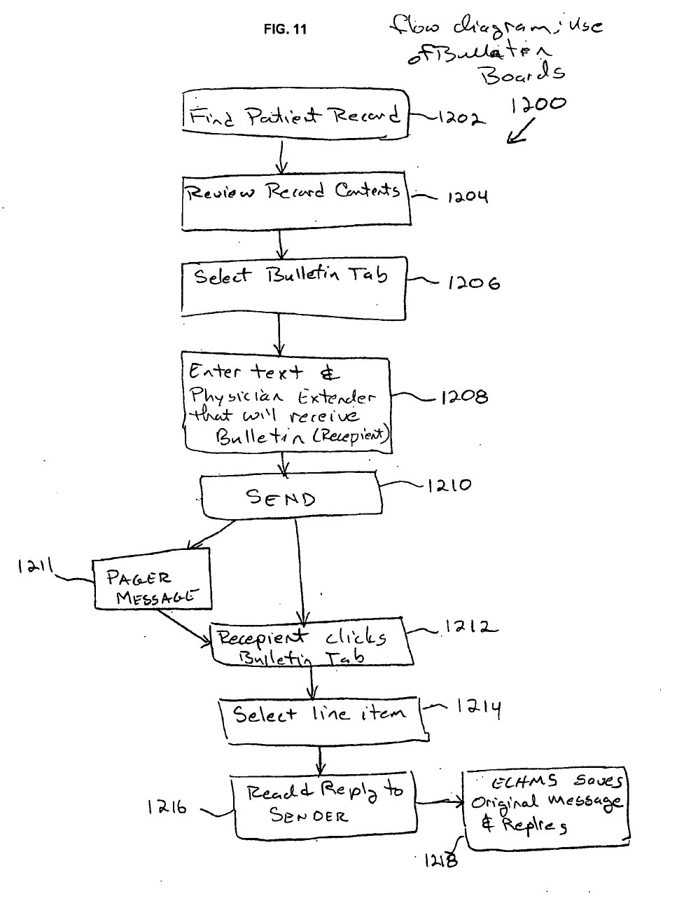

FIG. 11 illustrates a flow diagram of a method for using the doctor/physician extender bulletin board in an electronic care health management system in accordance with an embodiment of the present invention. Use of doctor/physician extender bulletin board 1100 begins with step 1202 when the doctor brings up a patient record by using Search from the left navigation bar, or by selecting the patient record box next to a patient's name on the calendar. In step 1204 the doctor reviews the contents of the record. If the doctor wishes to ask the physician extender a question about a treatment decision, or some other question related to that patient, the doctor selects the bulletins tab in step 1206. The bulletins tab provides a free text box in which the doctor enters the questions/comments. There is also a drop down selection box for the doctor to select the name of the physician extender who is to receive the message (step 1208). The doctor clicks “Send” (step 1210) when all of the comments/questions have been entered. The bulletin may be optionally escalated to a pager message (step 1211). The user can move from tab to tab within the record without losing or sending the comments/questions.

The physician extender is alerted to the presence of questions/comments by the “bulletins” button on the calendar page, beneath the “to do” list. The button changes color when there are messages. The physician extender clicks bulletins (step 1212) and a window appears with all messages by date and patient name. Selecting any line item (step 1214) brings up the patient's record, open to the “bulletins” tab. The physician extender reads the message and replies to the doctor (step 1216). On the doctor's home page, the “bulletins” button now changes color to indicate there are unread bulletins. The system records the running discussion commentary in the patient record (step 1218). Optionally, an additional feature may be integrated into use of doctor/physician extender bulletin board 1100 which comprises obtaining electronic signatures on patient records from the doctors.

ECHMS 100 has, as an inherent feature, a security system. The security system will be such that the medical director grants and controls access to users in the clinic. If the system administrator locks out the medical director for any reason, then all of the users in that clinic will also be locked out. The system administrator will not be granting access to any users other than the medical director for liability reasons. The medical director will be responsible for controlling clinic-level user access.

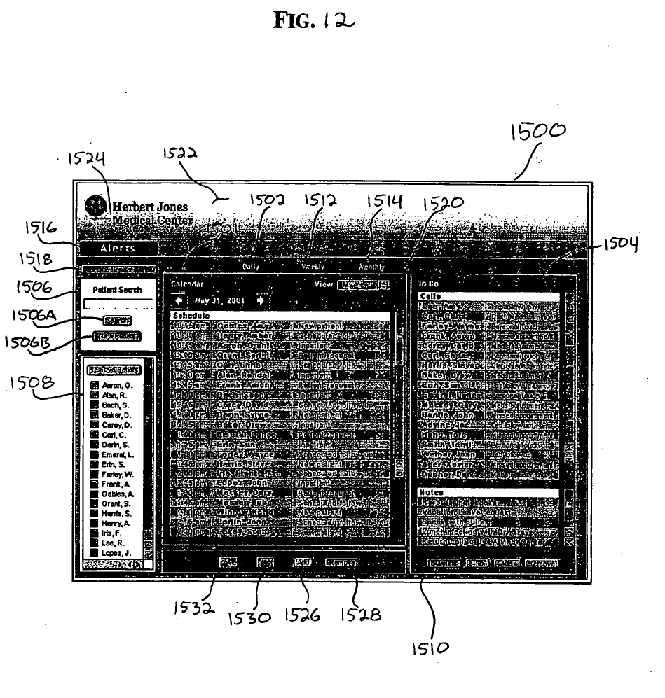

FIGS. 12-14 illustrate various user interfaces to ECHMS 100. FIG. 12 illustrates a physician extender's home page used in an electronic care health management system in accordance with an embodiment of the present invention.

Clinic interface 1500 was designed to provide maximum efficiency for time-pressed physician extenders and doctors. Specifically, the following tasks were identified as the most common for physician extenders: view and modify daily calendar; view and modify daily task list; access records for patients scheduled for appointments; manage patients by planning treatments; and prescribing medications.

Interface components such as the calendar and “to do” list are modeled on typical computer scheduling interfaces (for example, Microsoft Outlook®) to make them familiar and easy to learn. Colors are selected to be easy on the eyes since users will spend a lot of time looking at the screen. In addition, the color scheme can be easily modified to reflect the branding of each clinic that uses the system. Each clinic's logo appears at the top of the screen, cementing the notion that the system is part of the hospital, not just a third-party solution.

Clinic interface 1500 has been designed in order to separate common physician extender tasks into two areas: scheduling (this area encompasses calendar and “to do list” functionality, and furthermore does not require direct access to patient medical records); and patient management (this area includes all scenarios that involve direct interaction with the patient record). The physician extender's interaction with the system is primarily oriented towards scenarios involving scheduling. That is, the physician extenders mainly work with the calendar and the “to do” list, drilling down to patient management for appointments and tasks (calendar or “to do” list events). Once the event is complete, they return to the calendar or “to do” list to select a new event.

For these and other reasons, the home page has been designed around scheduling functionality by including the calendar and “to do” list components. The patient management page contains tabs that allow the physician extender to access patient data or functionality associated with the selected patient (for example, planning treatment or prescribing medications). The relationship between these two pages is illustrated in the following figure. (Please note that the tabs shown are examples only.)

Information for the physician extender interface flows from an event (an appointment or task on the “to do” list) to action on that event (pulling up a patient record for the appointment). The hub around which the interface is built is therefore the system components that display the event data (the calendar and “to do” List).

Interface elements for the physician extender pages are either persistent or page specific. Persistent page elements are designed to either aid application navigation, or represent functionality that must be accessible from multiple pages. The Patient Search panel is an example of a persistent page element.

Clinic interface 1500 contains several persistent page elements (PPE), which all appear on the left navigation bar. The first is Alerts PPE. Alerts PPE will change color (to red) to indicate that a patient's monitored data has reached a preset level (for example, a patient's weight has exceeded a set threshold). Selecting this element opens Alert Page which prominently displays the monitoring statistics that triggered the alert condition. Below these will be displayed all the statistics currently being captured from other monitoring devices.

The second persistent page element is patient search PPE. Patient search PPE contains text field for entering a patient's name. Once the name is entered (fully or partially) the user can select search button to find the patient record. Alternately, the user can create a new patient record by selecting new patient button.

The third persistent page element is patient list PPE. Patient list PPE is pre-populated by the system at the beginning of each work period (shift or day, depending on the clinic schedule) with patients who are associated with scheduled events. Clicking on the patient name opens the patent management window, with the selected patient's record.

The fourth persistent page element is calendar button PPE. Calendar button PPE is a static element that is simply a navigation element. When the home page is open, the button is de-selected (grayed out).

Page specific elements represent functionality that is only relevant to the page's intended use. “To do” list panel is an example of a page specific element. Page specific elements are discussed in detail with the descriptions of the page that contains them. A physician extender home page, discussed in detail below, is similar to clinic interface 1500 in that it has the same features as clinic interface 1500 except it is directed to the physician personally. That is, whereas clinic interface 1500 allows access to web pages for all the physicians in a clinic (for example, 8 physicians, 8 separate pages), A physician extender home page for physician A has only his (or her) web page visible.

The physician extender home page is composed of two page specific elements (PSE): calendar PSE 1501 and “to do” list PSE 1504. The purpose of these two page specific elements is to provide the physician extender with scheduling functionality, which is the focus of his or her workflow. Patient records can be accessed from this page either through events in calendar PSE 1501 or “to do” list PSE 1504, or through patient list persistent navigation element (PNE) 1508. Features of each page specific element and persistent navigation element for physician extender's home page 1500 will now be discussed in detail.

Calendar PSE 1501 has four major views, accessible via view button 1520: my view, doctor view, clinic view and all view. My view causes calendar PSE 1501 to display all the appointments/events for the user logged into the system. Doctor view causes calendar PSE 1501 to display all the appointments/events for the specific doctor. Clinic view causes calendar PSE 1501 to display all the appointments/events for the clinic (this may be filtered to show only public events, or events that the user has authorization to view). All view causes calendar PSE 1501 to overlay all of the appointments for the clinic, doctor and user. Calendar PSE 1501 can be viewed in day, week, or month view formats, and can be navigated using the arrow buttons.

Calendar PSE 1501 has several calendar-function buttons. Add calendar item button 1526 and remove calendar item button 1528 are buttons that can be used to edit the displayed calendar. Selecting add calendar item button 1526 will add an item at the time selected. Selecting remove calendar item button 1528 will delete the selected calendar item. Search calendar item button 1532 is represented by a magnifying glass. Lastly, clicking print calendar item button 1530 sends the currently displayed calendar to the printer (networked or local).

Title bar 1522 is a persistent navigation element. Title bar 1522 contains title logo 1524, which can be switched depending on the affiliation. “To do” list PSE 1504 is a page specific element. “To do” list 1504 contains a list of events that are not time specific. Events in this list can be added, edited, deleted, or checked off as completed. Or, if not completed or deleted, they will roll over to the following day's list.

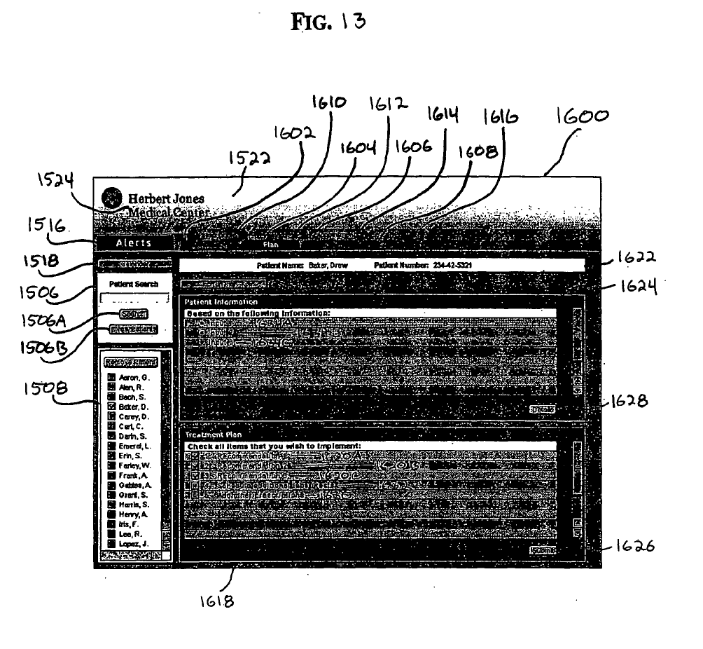

FIG. 13 illustrates a patient record page used by an electronic care health management system in accordance with an embodiment of the present invention. In FIG. 15, if a patient name is double clicked, then patient record page 1600 is displayed. Patient record page 1600 is populated with data from the CHF clinic record of the selected patient. Patient data is organized into tabs that represent data groups (for example, meds tab 1612 represents the patients' medication history) or workflows that relate to the patient (for example, plan tab 1604 allows the physician extender or doctor to create a treatment plan).

One of the important design constraints regarding patient record page 1600 is that because there is a wide range of patient data spread over many tabs, it is possible for the physician extender or doctor to attempt to save a record with incomplete information. For this reason, the record will be verified prior to committing the record to the database. The user will be queried to see if he or she does indeed want to save an incomplete record. If so, the record will be saved. If not, however, the tabs containing incomplete data will change color to indicate that they contain fields that must be completed prior to saving the record.

There are eight (8) navigation tabs (Tab) present that contain functionality related to the open patient record. ECHMS 100 keeps track of the status of the open record, and if another patient record is opened before all required fields have been updated, will remind the user that data needs to be entered before the record can be saved.

Plan tab 1604 is comprised of patient information field 1624 and treatment field 1618. Patient information field 1624 displays the patient's name and number to identify the record being reviewed plus the data that the protocol is using to build a recommendation. This is shown as input data 1634A-C. If this data is inaccurate, the user can select the appropriate tab to navigate to the section of the patient record that must be updated. Run plan button 1620 will, when clicked, run the appropriate treatment protocol based on the inputs (i.e., input data 1634A-C) in patient information field 1634. Details button 1628 displays protocol details, when clicked. And, submit button 1626 submits the plan (those recommendations 1620A-C that have been checked) and opens a wizard to allow the user to specify medications to meet the recommendations.

Treatment plan field 1618 displays recommendations 1620 (in this case, 1620A-C). This list of recommendations 1620 are based on the relevant protocol. The user can select recommendations to implement, and then click submit button 1626 to specify medications. Treatment plan field 1618 also contains line items that may be selected if the user wants to add prescriptions (prescription 1630) or schedule additional labs (schedule lab/test 1632).

Another tab in patient record page 1600 is patient history tab 1602. In patient history tab 1602 users may view and edit the CHF CPR. Tab 1604 has been described in detail above. Plan tab 1604 is used to create a treatment. The treatment plan is generated by a protocol rules engine and based on data in the CHF CPR. The page is divided into two sections, the data that the rules engine uses to generate the recommendations (patient information field 1624), and the recommendations themselves (treatment plan field 1618). Plan tab 1604 is actually the starting point for the plan treatment scenario. Lab tab 1606 is used to enter and view lab results. Prescriptions tab 1610 is used to enter prescriptions and send them to a pharmacy via facsimile or other electronic means, such as email or EDI.

Meds tab 1612 is used to view medication history. Appointments tab 1614 gives the physician extender access to the selected patient's calendar. The physician extender can then schedule appointments or lab visits to that calendar. Physical examination (PE) Tab 1616 is used to record the findings from physical examinations.

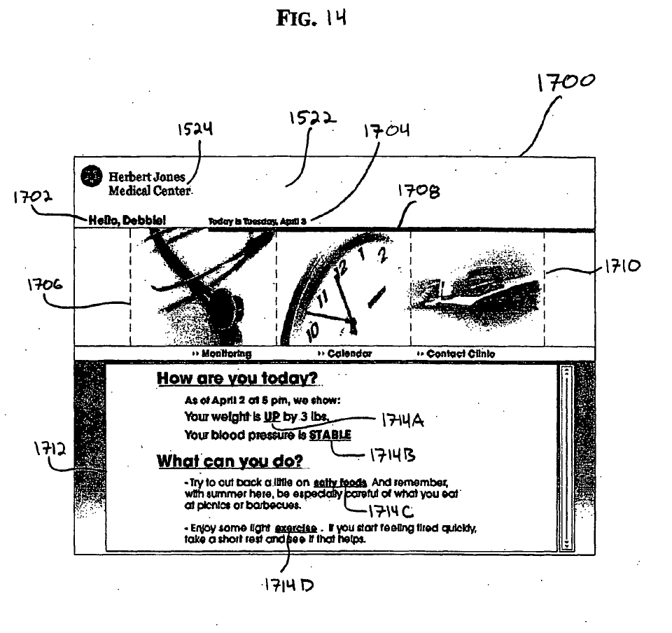

FIG. 14 illustrates a patient interface in an electronic care healthcare management system in accordance with an embodiment of the present invention. Patient interface 1700 looks very different than the interface screens used by other users of ECHMS 100. Rather than displaying vast quantities of data, patient interface 1700 is simple and engaging; it is designed to encourage patients to log on and monitor their health, learn more about their disease, and stay in touch with the CHF clinic. By creating a dynamic interface, patients will receive feedback from the data collected by their monitoring devices. The constant interaction will hopefully encourage patients to manage their diet and lifestyle habits, because they can see the impact of their actions on a daily basis.

Like the clinic screens, patient interface 1700 can easily be branded according to the clinic implementing the system (vis-a-vis title bar 1522 and title logo 1524). The URL the patient types and enters directs patients to monitoring page 1712 that functions as the home page. Logon (username and password) is required for access to patient interface 1700. Once patient interface 1700 opens, the patient's name appears, along with the date and time of the present logon and the most previous logon activity. This occurs so that the patient can see how recent the last monitoring results are.

There are three large panels used as navigation buttons, and these direct users to the three main parts of patient interface 1700: monitoring panel 1706, calendar panel 1708 and contact clinic panel 1710. Clicking on monitoring panel 1706 directs the patient to monitoring page 1712. Clicking on contact clinic panel 1710 directs the patient to a contact clinic page. Contact clinic page contains contact information that may contain insurance contact information, telephone and/or pager contact information (for the insurance company, clinic and physicians and perhaps other parties) and e-mail contact information, again for the clinic, physician, insurance company and perhaps others). Clicking on calendar panel 1708 directs the patient to a patient calendar page. The patient calendar page is a calendar, with multiple viewing formats that enable the patient to see when clinic appointments, lab tests and educational seminars are scheduled.

Monitoring panel 1706 may contain hot links in order to provide additional information to the patients. For example, as seen in FIG. 17, monitoring page 1706 has four hot links: UP hot link 1714A, STABLE hot link 1714B, Salty foods hot link 1714C and Exercise hot link 1714D. UP hot link 1714A provides a link to a page with information about why a patient's weight may have increased and what they can do about it. STABLE hot link 1714B provides a link to a page congratulating the patient on their good blood pressure. This may provide motivation to the patient to keep to their good habits. Salty foods hot link 1714C provides a link to a page listing salty foods that the patient should avoid. Exercise hot link provides a link to a page with light exercise ideas for the patient.

Clicking on calendar panel 1708 directs the patient to a calendar page, which provides the patient with medical appointments that have been set by the CHF clinic. The patient or clinic can also enter medication schedules and other reminders. Clicking on contact clinic panel 1710 directs the patient to contact clinic page, which provides phone numbers and reminders of what to do in case of emergency. Contact clinic page can include email links if the clinic wants to receive patient email.

The present invention has been described with reference to certain exemplary embodiments. However, it will be readily apparent to those skilled in the art that it is possible to embody the invention in specific forms other than those of the exemplary embodiments described above. This may be done without departing from the spirit of the invention. The exemplary embodiments are merely illustrative and should not be considered restrictive in any way. The scope of the invention is defined by the appended claims, and their equivalents, rather than the proceeding description. Rather than the preceding description, and all variations and equivalents which fall within the range of the claims are intended to be embraced therein.

What is claimed is:

1. A method for managing an electronic based healthcare facility, comprising:
finding a clinic patient record for an established patient, or creating a new clinic patient record for a new patient;
recording patient information into the patient's clinic patient record;
submitting the patient's clinic patient record to a rules engine wherein the patient's clinic patient record is run according to a rules engine protocol;
verifying that the recorded patient's information is correct and correcting the recorded patient's information if it is not correct;
prescribing a course of treatment based on the rules engine recommendations; and
fulfilling the prescribed course of treatment.

2. The method for managing an electronic based healthcare facility according to claim 1, wherein the step of finding a clinic patient record for an established patients comprises:
determining whether an existing patient is on a patient record navigation bar;
searching for the patient if the patient is not on the patient record navigation bar;
entering the patient's name into a patient search field;
selecting a search operation; and
reading the patient's patient management page.

3. The method for managing an electronic based healthcare facility according to claim 1, wherein the step of finding a clinic patient record for an established patients comprises:
determining whether an existing patient is on a patient record navigation bar;
selecting the patient if the patient is on the patient record navigation bar; and
reading the patient's patient management page.

4. The method for managing an electronic based healthcare facility according to claim 1, wherein the step of recording patient information into the patient's clinic patient record comprises:
obtaining updated information from the patient;
entering the updated information in to the appropriate location in a patient's patient management page; and
saving the patient's patient management page with the updated information into a clinical patient record.

5. The method for managing an electronic based healthcare facility according to claim 1, wherein the step of creating a new clinic patient record comprises:
obtaining external referral information about a patient;
obtaining general information about the patient;
obtaining first visit patient enrollment information; and
recording the external referral information, general information, and first visit patient enrollment information into the clinical patient record.

6. The method for managing an electronic based healthcare facility according to claim 4, wherein the step of obtaining external referral information about a patient comprises:
adding a patient to a clinical patient record; and
entering the patient's referral information to the clinical patient record.

7. The method for managing an electronic based healthcare facility according to claim 4, wherein the step of obtaining general information about the patient comprises:
entering general information about the patient to the clinical patient record; and
scheduling an appointment for the patient at a healthcare facility.

8. The method for managing an electronic based healthcare facility according to claim 4, wherein the step of obtaining first visit patient enrollment information comprises:
obtaining enrollment information from the patient;
obtaining insurance and/or billing information from the patient;
obtaining patient goals from the patient;
obtaining patient data from the patient;
providing initial education to the patient;
reviewing the patient goals with the patient; and
recording the enrollment information, patient goals and patient date into the clinical patient record.

9. The method for managing an electronic based healthcare facility according to claim 4, wherein the step of obtaining enrollment information from the patient comprises:
obtaining patient demographic information from the patient.

10. The method for managing an electronic based healthcare facility according to claim 1, wherein the step of submitting the patient's clinic patient record to a rules engine wherein the patient's clinic patient record is run according to a rules engine protocol comprises:
evaluating the information contained in a patient's clinical patient record against a set of medical protocols;
generating one or more diagnoses based on the patient's record in the clinical patient record;
generating one or more recommendations based on the one or more patient's diagnoses, one or more insurance company's formularys and the patient's clinical patient record; and
issuing the one or more recommendations.

11. The method for managing an electronic based healthcare facility according to claim 10, wherein the step of generating one or more recommendations based on the one or more patient's diagnoses, one or more insurance company's formularys and the patient's clinical patient record comprises:
comparing an insurance company's prescription formulary against a prescription recommendation generated by the rules engine protocol;
comparing an insurance company's lab and test formulary against a lab and test recommendation generated by the rules engine protocol; and
comparing an insurance company's calendar formulary against a calendar recommendation generated by the rules engine protocol.

12. The method for managing an electronic based healthcare facility according to claim 10, wherein the recommendation is selected from the group consisting of prescription recommendations, lab and test recommendations, calendar recommendations and lifestyle/diet issues recommendations.

13. The method for managing an electronic based healthcare facility according to claim 1, wherein the step of proscribing a course of treatment based on the rules engine recommendations comprises:
reviewing the recommendations returned by the rules engine protocols;
selecting recommendations for further review;
entering text for any recommendations that were not selected for further review; and
verifying the selected recommendations for correctness.

14. The method for managing an electronic based healthcare facility according to claim 13, wherein the step of submitting the verified, selected recommendations to the rules engine protocol to fulfill the recommendations comprises:
receiving a detailed set of recommendations;
reviewing the detailed set of recommendations returned by the rules engine protocol;
modifying the one or more of the detailed set of recommendations returned by the rules engine protocol if necessary until correct;
verifying that all recommendations have been reviewed for correctness;
causing the recommendations to be accomplished;
updating the patient's clinical patient record to reflect the recommendations and the accomplishing of the recommendations; and
issuing a billing and/or claim report form.

15. The method for managing an electronic based healthcare facility according to claim 14 wherein the recommendations is selected from a group consisting of a prescription recommendation, lab and test recommendation, calendar recommendation and a lifestyle/diet issues recommendation.

16. A method for calendar and event scheduling for use in an electronic care health management system comprising
selecting a calendar and a calendar view;
reviewing calendar view data;
updating the calendar with new calendar information.

17. The method for calendar and event scheduling for use in an electronic care health management system according to claim 16, wherein the step of updating the calendar with new calendar information comprises:
deciding whether or not to create a new event;
entering new event details if a new event is to be created;
editing an existing calendar event with existing calendar event new details if a new event is not to be created; and
posting either the existing calendar event with existing calendar event new details or the new event details.

18. A method for entering patient encounter data into a clinical patient record for use in an electronic care health management system, comprising:
opening a patient record from a clinical patient record;
selecting a patient history tab on the patient record for a patient, thereby opening a patient encounter form; and
entering new information for the patient in the patient's patient encounter form, or, editing an existing patient encounter form for the patient.

19. The method for entering patient encounter data into a clinical patient record for use in an electronic care health management system according to claim 18, wherein the step of entering new information for the patient in the patient's patient encounter form comprises:
entering the new information for the patient manually through use of a telephone encounter form;
saving the new information contained in the telephone encounter form into the clinical patient record for the patient; and
optionally printing the telephone encounter form.

20. The method for entering patient encounter data into a clinical patient record for use in an electronic care health management system according to claim 18, wherein the step of entering new information for the patient in the patient's patient encounter form comprises
entering the new information for the patient automatically through use of a voice annotation apparatus;
dictating the new information for the patient into a temporary voice annotation file;
saving the new information contained in the voice annotation file into the clinical patient record for the patient; and
optionally editing and printing the voice annotation file.

21. The method for entering patient encounter data into a clinical patient record for use in an electronic care health management system according to claim 18, wherein the step of editing an existing patient encounter form for the patient comprises:
scrolling or searching to find a previously created patient encounter form for the patient;
selecting the patient encounter form;
editing the patient encounter form with new information; and
saving the edited patient encounter form to the clinical patient record for the patient, and optionally printing the edited patient encounter form.

22. A method for utilizing a doctor/physician extender bulletin board in an electronic care health management system comprising:
opening a patient's patient record from a clinical patient record;
reviewing the patient's patient record selecting a bulletin tab on the patient record for a patient, thereby opening a bulletin form;
entering text regarding the patient in the patient's patient bulletin form;
selecting one or more doctors/physician extenders to receive the patient's patient bulletin form; and
forwarding the patient's patient bulletin form to the one or more doctors/physician extenders.

23. The method for utilizing a doctor/physician extender bulletin board in an electronic care health management system according to claim 22 further comprising:
selecting a paging option, to optionally page the one or more doctors/physician extenders that a patient's patient bulletin form is being sent to the to the one or more doctors/physician extenders.

24. The method for utilizing a doctor/physician extender bulletin board in an electronic care health management system according to claim 22 further comprising:
receiving a pager regarding a forwarded patient's patient bulletin form or electronic notification that a forwarded patient's patient bulletin form has been received for the one or more doctors/physician extenders;
selecting a bulletin tab on the recipient's calendar page, thereby opening a line item list of all received bulletin forms;
selecting a line item corresponding to a particular patient's patient bulletin form;
reviewing the received patient's patient bulletin form, and optionally responding if desired; and
saving the received patient's patient bulletin form and any replies sent by the receiving one or more doctors/physician extenders.

25. An electronic healthcare management system, comprising:
a data center, adapted to comprise a first network server and data storage, the network server hosting a rules engine, and providing for the operation of various web-based interfaces to the electronic healthcare management system including a system administration interface;
a healthcare facility, adapted to comprise a second network server and providing for the operation of various web-based interfaces and functions to the electronic healthcare management system;
a patient, adapted to comprise an intelligent device which provides for remote monitoring and an education and support function;
other information systems; and
a network providing for communications between the data center, healthcare facility, patient, and other information systems.

26. The electronic healthcare management system according to claim 25, wherein the healthcare facility further comprises:
a manual data entry function, a clinical patient record function, a voice annotation function, an outcomes function, a local clinic administration function, a routine office tools function, an alerts function, a report support function; medications, recommendations function and a billing function.

27. The electronic healthcare management system according to claim 25, wherein the other information systems includes at least the following:
one or more pharmacies;
one or more hospitals;
one or more laboratories/diagnostic services;
one or more federal agencies/departments; and
one or more state and local agencies/departments.

28. The electronic healthcare management system according to claim 25, wherein the rules engine and business logic comprises:
a set of decision making instruction code adapted to review a patient's clinical patient record, current suggestions by an attending physician, established insurance company formularys with an adaptable base of medical knowledge to create treatment recommendations for the patient.

29. The electronic healthcare management system according to claim 25, wherein the rules engine and business logic further comprises:
an adaptation to learn from the healthcare facility doctors/physician extenders to adapt the base of medical knowledge to incorporate local knowledge.

30. The electronic healthcare management system according to claim 25, wherein the rules engine and business logic further comprises:
an adaptation to incorporate shared information between billing, insurance formulary compliance, scheduling of lab/diagnostic recommendations, prescription recommendations, calendar recommendations and lifestyle/diet issues recommendations to provide an enhanced complete healthcare system sharing information seamlessly.

31. The method for managing an electronic based healthcare facility according to claim 1, wherein the step of fulfilling the proscribed course of treatment comprises:
submitting the verified, selected recommendations to the rules engine protocol.
